(19) United States
(12) Patent Application Publication
(10) Pub. No.: US 2012/0157421 A1
(43) Pub. Date: Jun. 21, 2012

(54) 17Beta-Alkyl-17Alpha-oxy-estratrienes

(75) Inventors: Rolf Bohlmann, Berlin (DE); Nikolaus Heinrich, Berlin (DE); Jan Hübner, Berlin (DE); Georg Kettschau, Berlin (DE); Hermann Künzer, Berlin (DE); Philip Lienau, Berlin (DE); Michael Gerisch, Wuppertal (DE); Silke Köhr, Berlin (DE); Dieter Lang, Velbert (DE); Karsten Denner, Berlin (DE); Michael Sander, Hoffnungsthal (DE); Jens Hoffmann, Muhlenbeck (DE); Tim Wintermantel, Koln (DE)

(73) Assignee: BAYER PHARMA AKTIENGESELLSCHAFT, Berlin (DE)

(21) Appl. No.: 13/376,194

(22) PCT Filed: May 26, 2010
(86) PCT No.: PCT/EP2010/003204

§ 371 Date: Feb. 29, 2012

(30) Foreign Application Priority Data
Jun. 4, 2009 (EP) 09075249.4

(51) Int. Cl. A61K 31/58; A61K 31/56; C07J 3/00; A61P 5/30; C07J 31/00; C07J 41/00; C07J 43/00

(52) U.S. Cl. 514/176; 552/610; 514/182; 540/107

(57) ABSTRACT

The invention relates to 17β-alkyl-17α-oxy-oestratrienes of the formula (I), to processes for their preparation, to the use of the 17β-alkyl-17α-oxy-oestratrienes for preparing pharmaceuticals and to pharmaceutical preparations comprising these compounds.

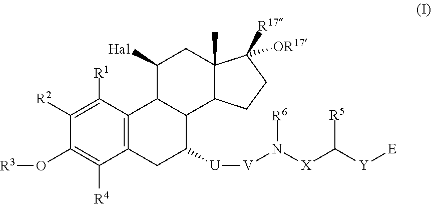

The invention relates to 17β-alkyl-17α-oxy-oestratrienes and to processes for their preparation, to the use of the 17β-alkyl-17α-oxy-oestratrienes for preparing pharmaceuticals and to pharmaceutical preparations comprising these compounds.

The compounds according to the invention have antioestrogenic action, i.e. these substances exert inhibiting actions relative to oestrogens. Such substances have already been described extensively.

For example, compounds that have an antioestrogenic action are known from EP 0 138 504 B1. These are essentially oestra-1,3,5(10)-triene derivatives which are substituted in the 3-position inter alia with hydroxyl or alkoxy, in the 17α-position with hydroxyl and in the 17β-position inter alia with hydrogen or alkyl. These compounds furthermore have, in the 7α-position, an alkyl side chain which may be partially fluorinated and which may be interrupted inter alia by amido, amino, amine N-oxide, oxy, sulphanyl, sulphinyl and/or sulphonyl groups.

WO 99/33855 A1 describes 11β-halo-7α-substituted oestra-1,3,5(10)-trienes which may have hydroxyl groups in the 3- and 17-positions. The 7α-side chain is a partially fluorinated, optionally unsaturated hydrocarbon chain which is interrupted by an amine nitrogen atom or by a sulphanyl, sulphinyl or sulphonyl group.

Other compounds are described in WO 98/07740 A1. These are substituted 7α-(ξ-aminoalkyl)oestra-1,3,5(10)-trienes. In the 3-position, these compounds preferably have a hydroxyl, methoxy or acetyloxy group, and in the 17β- and/or 17α-position they preferably have a methyl or trifluoromethyl group. The 11β-position is preferably occupied by a fluorine atom and the 7α-position by an alkyl side chain which is terminally at least partially fluorinated and which is interrupted by an amine nitrogen atom and by a sulphanyl, sulphinyl or sulphonyl group.

WO 97/45441 A1 discloses 7α-(5-methylaminopentyl)oestra-1,3,5(10)-trienes which have hydroxyl groups in the 3-position and in the 17α-position. The 17β-position may be occupied by a methyl or ethynyl group. Furthermore, the 2-position of the oestratriene skeleton may also be substituted by a fluorine atom.

It has been found that, on administration, the known compounds form a variety of biologically highly active metabolites. The formation of these metabolites leads to unwanted actions and thus an uncontrollable activity spectrum. In particular, side effects may occur, or the desired primary action (antioestrogenic action) becomes uncontrollable by the spontaneous formation of these metabolites. Moreover, the compatibility of the known compounds on oral administration is unsatisfactory. In particular, it has been found that the known compounds promote the accumulation of alveolar macrophages.

Common to all antioestrogens of the prior art is an unsatisfactory oral bioavailability. Moreover, the antioestrogens of the prior art interact with cytochrome P450 oxidases. The structurally closest prior art form compounds of WO 03/045972 which differ from the compounds according to the invention in that the group in position 17 which is different from hydrogen is not attached in the α-position, but in the β-position, and the oxygen-containing radical likewise bonded in position 17 is attached to the oestratriene skeleton not in the β-position, but in the α-position.

Up to now, the prior art led away from 17β-alkyl-17α-oxy-oestratrienes, as there were numerous indications that these compounds had no high affinity to the oestrogen receptor, and accordingly neither oestrogenic nor antioestrogenic effects were anticipated (J. Med. Chem. 1979, 22(12), 1538ff.). Even if the person skilled in the art had dismissed this, he would have had the problem of finding a synthesis route for 17β-alkyl-17α-oxy-oestratrienes, as the prior art describes only routes which are complicated and of little promise.

Based on this prior art, it is an object of the present invention to provide further antioestrogen compounds which form few, if any, biologically active metabolites. Moreover, on oral administration, the compounds sought should have improved bioavailability, thus reducing patient stress. This increases patient compliance.

Surprisingly, and dismissing the preconceptions of the person skilled in the art, it has now been found that the 17β-alkyl-17α-oxy-oestratrienes according to the invention have anti-oestrogenic properties and considerably improved bioavailability. This was unexpected.

The object according to the invention is achieved by 17β-alkyl-17α-oxy-oestratrienes of the general formula (I)

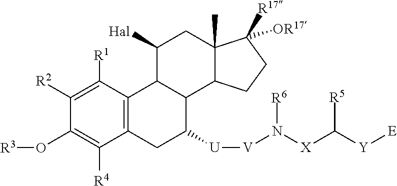

in which
Hal represents fluorine or chlorine which is attached in the 11β-position to the oestratriene skeleton; and
R¹, R² and R⁴ independently of one another, represent hydrogen, fluorine, chlorine or bromine, and
R³ represents hydrogen or a C₁-C₄-alkyl or C₁-C₄-alkanoyl radical, and
R^{17′} represents hydrogen or a C₁-C₄-alkyl or C₁-C₄-alkanoyl radical,
R^{17″} represents an optionally mono- or polyfluorinated C₁-C₄-alkyl, C₂-C₄-alkenyl or C₂-C₄-alkynyl radical,
   where R^{17′}—O is attached in the 17α-position and R^{17″} is attached in the 17β-position to the oestratriene skeleton; and
U represents a straight-chain or branched C₁-c₁₃-alkylene, C₂-C₁₃-alkenylene or C₂-C₁₃-alkynylene radical, or
   represents the group A-B, where
   A is attached to the oestratriene skeleton and is a benzylidene radical attached via —CH₂— to the oestratriene skeleton, is a phenylene radical or is a C₁-C₃-alkylene-phenylene radical attached via the alkyl group to the oestratriene skeleton and
   B is a straight-chain or branched C₁-C₁₃-alkylene, C₂-C₁₃-alkenylene or C₂-C₁₃-alkynylene radical and
   where A and B may also be attached to one another via an oxygen atom,
V represents a methylene or a —C(O)— group and
X represents a bond or a C₁-C₃-alkylene group and
R⁵ represents hydrogen or a C₁-C₄-alkyl, C₂-C₄-alkenyl or C₂-C₄-alkynyl radical
R⁶ represents hydrogen or the group —CH₂—R⁷ or C(O)—R⁷, in which R⁷ represents hydrogen or a straight-chain or branched nonfluorinated or at least partially fluorinated C₁-C₆-alkyl, C₂-C₆-alkenyl or C₂-C₆-alkynyl radical which may be mono- or polysubstituted by hydroxyl, or
R⁵ and R⁶ together with X and the nitrogen atom of the side chain form a 4- to 6-membered heterocyclyl ring which, in addition to the nitrogen atom of the side chain, may have a further heteroatom and/or may contain a carbonyl group,
Y represents a C₅-C₈-alkylene group,
E represents a C₁-C₄-perfluoroalkyl radical or represents a phenyl radical which is mono- to pentasubstituted by halogen or —CF₃,
   and their enantiomers and diastereomers, their salts, solvates and salts of the solvates.

The application is based on the following definitions:

### Cₙ-Alkyl:

Cₙ-Alkyl represents a straight-chain or branched saturated monovalent hydrocarbon radical having n carbon atoms.

### Cₙ-Alkylene:

Cₙ-Alkylene represents a straight-chain or branched saturated divalent hydrocarbon radical having n carbon atoms.

### Cₙ-Alkenylene:

Cₙ-Alkenylene represents a straight-chain or branched divalent hydrocarbon radical having n carbon atoms and at least one double bond.

### Cₙ-Alkynylene:

Cₙ-Alkynylene represents a straight-chain or branched divalent hydrocarbon radical having n carbon atoms and at least one triple bond.

### Cₙ-Alkenyl:

Cₙ-Alkenyl represents a straight-chain or branched monovalent hydrocarbon radical having n carbon atoms and at least one double bond.

### Cₙ-Alkynyl:

Cₙ-Alkynyl represents a straight-chain or branched monovalent hydrocarbon radical having n carbon atoms and at least one triple bond.

### Cₙ-Alkylcarbonyl=Cₙ-Alkanoyl:

Cₙ-Alkylcarbonyl represents the group —C(O)—Cₙ-alkyl.

In general, n is from 1 to 6, preferably from 1 to 4 and particularly preferably from 1 to 3.

The following radicals may be mentioned by way of example and by way of preference: Acetyl and propanoyl.

### Heteroatoms:

Heteroatoms are to be understood as meaning oxygen, nitrogen or sulphur atoms.

### Heteroaryl

Heteroaryl is a monovalent aromatic mono- or bicyclic ring system having at least one heteroatom different from carbon. Heteroatoms which may be present are nitrogen atoms, oxygen atoms and/or sulphur atoms. The binding valency may be at any aromatic carbon atom or at a nitrogen atom.

### Heterocyclyl

For the purpose of the invention, heterocyclyl is a fully or partially hydrogenated heteroaryl (fully hydrogenated heteroaryl=saturated heterocyclyl), i.e. a nonaromatic mono- or bicylic ring system having at least one heteroatom different from carbon or a hetero group. Heteroatoms which may be present are nitrogen atoms, oxygen atoms and/or sulphur atoms. The binding valency may be at any carbon atom or at a nitrogen atom.

A monocyclic heterocyclyl ring in accordance with the present invention may have from 4 to 6 ring atoms.

Heterocyclyl rings having 4 ring atoms (4-membered) include, for example, azetidinyl.

Heterocyclyl rings having 5 ring atoms (5-membered) include, for example, the rings pyrrolidinyl, imidazolidinyl, pyrazolidinyl and pyrrolinyl.

Heterocyclyl rings having 6 ring atoms include, for example, the rings piperidinyl, piperazinyl, morpholinyl and thiomorpholinyl.

### Halogen

The term halogen includes fluorine, chlorine, bromine and iodine.

Preference is given to fluorine.

In the general formula (I), Hal may represent fluorine or chlorine which is attached in the 11β-position to the oestratriene skeleton.

Preferably, Hal represents a fluorine atom.

In the general formula (I), R¹, R² and R⁴ may independently of one another represent hydrogen, fluorine, chlorine or bromine.

Preferably, R¹, R² and R⁴ independently of one another represents hydrogen, chlorine or bromine.

Particularly preferably, R¹ represents hydrogen, R² represents hydrogen or chlorine and R⁴ represents hydrogen, chlorine or bromine.

Most preferably, R¹, R² and R³ represent hydrogen.

In the general formula (I), R³ may represent hydrogen or a C₁-C₄-alkyl or C₁-C₄-alkanoyl radical.

Preferably, R³ represents hydrogen, methyl, ethyl, n-propyl, isopropyl, n-butyl, isobutyl or tert-butyl or a corresponding alkanoyl (acetyl, propionyl, butanoyl).

Particularly preferably, R³ represents hydrogen or a methyl or acetyl radical.

Most preferably, R³ represents hydrogen.

In the general formula (I), R⁵ may represent hydrogen or a C₁-C₄-alkyl, C₂-C₄-alkenyl or C₂-C₄-alkynyl radical.

R⁵ preferably represents hydrogen or a C₁-C₃-alkyl radical.

R⁵ particularly preferably represents hydrogen.

In the general formula (I), R⁶ may represent hydrogen or the group —CH₂—R⁷ or C(O)—R⁷ in which R⁷ represents hydrogen or a straight-chain or branched nonfluorinated or at least partially fluorinated C₁-C₆-alkyl, C₂-C₆-alkenyl or C₂-C₆-alkynyl radical which may be mono- or polysubstituted by hydroxyl, or

R⁶ preferably represents hydrogen or —CH₂—R⁷ in which R⁷ represents in particular hydrogen or a methyl or ethyl radical.

R⁶ particularly preferably represents a methyl group.

In the general formula (I), R⁵ and R⁶ may alternatively together with X and the nitrogen atom of the side chain form a 4- to 6-membered heterocyclyl ring which, in addition to the nitrogen atom of the side chain, may have a further heteroatom and/or may contain a carbonyl group. R⁵ and R⁶ together with the nitrogen atom of the side chain preferably form a 5-membered heterocyclyl ring which, in addition to the nitrogen atom of the side chain, may have a further heteroatom and/or may contain a carbonyl group.

R⁵ and R⁶ together with the nitrogen atom of the side chain particularly preferably form a pyrrolidine ring.

In the general formula (I), R^{17′} may represent hydrogen or a C₁-C₄-alkyl or a C₁-C₄-alkanoyl radical.

In the general formula (I), R^{17″} may represent an optionally mono- or polyfluorinated C₁-C₄-alkyl-, C₂-C₄-alkenyl or C₂-C₄-alkynyl radical.

R^{17′}—O is attached in the 17α-position and R^{17″} in the 17β-position to the oestratiene skeleton.

R^{17′} and R^{17″} are in particular methyl, ethyl, n-propyl, isopropyl, n-butyl, isobutyl and tert-butyl.

Hydrogen, acetyl, propionyl and butanoyl are additionally preferred for R^{17′}.

Ethynyl, 1-propynyl, 2-propynyl, 1-butynyl, 2-butynyl and 3-butynyl, and also trifluoromethyl, pentafluoroethyl, heptafluoropropyl and nonafluorobutyl are furthermore preferred for R^{17″}.

R^{17′} particularly preferably represents hydrogen, a methyl radical or an acetyl radical.

R^{17″} particularly preferably represents a methyl, ethynyl or trifluoromethyl radical.

R^{17′} most preferably represents hydrogen.

R^{17″} most preferably represents a methyl radical.

In the general formula (I), U may represent a straight-chain or branched C₁-C₁₃-alkylene, C₂-C₁₃-alkenylene or C₂-C₁₃-alkynyl radical, or

may represent the group A-B, where
A is attached to the oestratriene skeleton and is a benzylidene radical attached via —CH₂— to the oestratriene skeleton, is a phenylene radical or is a C₁-C₃-alkylene-phenylene radical attached via the alkyl group to the oestratriene skeleton and
B is a straight-chain or branched C₁-C₁₃-alkylene, C₂-C₁₃-alkenylene or C₂-C₁₃-alkynylene radical and
where A and B may also be attached to one another via an oxygen atom,
U may in particular be a straight-chain or branched alkylene radical.

Preference is given to a methylene, ethylene, propylene, butylene, pentylene, hexylene, heptylene, octylene, nonylene, decylene, undecylene, dodecylene or tridecylene radical.

Particularly preferably, U represents —(CH₂)ₚ—, where p is an integer from 2 to 10.

Especially preferably, U is a butylenes, pentylene, hexylene or heptylene radical.

Most preferably, U is an n-butylene radical, i.e. in the formula —(CH₂)ₚ— for U, p=4.

In the general formula (I), V may represent a methylene or a C(O) group.

V in particular represents a methylene group. Thus, in a most preferred embodiment, the grouping U-V may be n-pentylene.

In the general formula (I), X may represent a bond or a C₁-C₃-alkylene group.

Preferably, X is a bond or a methylene group.

Particularly preferably, X is a bond.

In the general formula (I), Y may represent a C₅-C₈-alkylene group.

Preferably, Y is a bond or a C₅-C₇-alkylene group.

Particularly preferably, Y is an n-pentylene or n-hexylene group.

In the general formula (I), E may represent a C₁-C₄-perfluoroalkyl radical or represent a phenyl radical which is mono to pentasubstituted by halogen or —CF₃.

E preferably represents —CF₃, —C₂F₅, —C₃F₇, —C₄F₉, represents phenyl which is mono- to trisubstituted by halogen and/or trifluoromethyl.

E particularly preferably represents —C₂F₅, —C₃F₇, —C₄F₉ or represents a trifluoromethylphenyl radical.

E most preferably represents —C₂F₅.

A preferred subgroup of compounds is formed by compounds according to formula (I) in which
Hal represents fluorine, and
R¹, R² and R⁴ independently of one another represent hydrogen, chlorine or bromine, and
R³ represents hydrogen, a methyl or acetyl radical, and
R⁵ represents hydrogen or a C₁-C₃-alkyl radical, and
R⁶ represents hydrogen or —CH₂—R⁷, in which R⁷ represents hydrogen or a methyl or ethyl radical, or
R⁵ and R⁶ together with the nitrogen atom of the side chain form a 5-membered heterocyclyl ring which, in addition to the nitrogen atom of the side chain, may have a further heteroatom and/or may contain a carbonyl group, and
R^{17′} represents hydrogen, a methyl radical or acetyl radical, and
R^{17″} represents a methyl, ethynyl or trifluoromethyl radical, and
U represents a butylene, pentylene, hexylene or heptylene radical, and
V represents a methylene group, and
X represents a bond or a methylene group, and
Y represents a bond or a C₅-C₇-alkylene group, and
E represents —C₂F₅, —C₃F₇, —C₄F₉ or represents a trifluoromethylphenyl radical,
   and their enantiomers and diastereomers, their salts, solvates and salts of the solvates.

A most preferred subgroup of compounds is formed by compounds according to formula (I) in which

R¹ represents hydrogen, and
R² represents hydrogen or chlorine, and
R⁴ represents hydrogen, chlorine or bromine, and
R³ represents hydrogen, and
Hal represents fluorine, and
R⁵ represents hydrogen and R⁶ represents a methyl group or
R⁵ and R⁶ together with the nitrogen atom of the side chain form a pyrrolidine ring, and
R^{17′} represents hydrogen, and
R^{17″} represents a methyl radical, and
U represents an n-butylene radical, and
V represents a methylene group, and
X represents a bond, and
Y represents an n-pentylene or n-hexylene group, and
E represents —C₂F₅,
and their enantiomers and diastereomers, their salts, solvates and salts of the solvates.

In accordance with the present invention, pharmacologically acceptable acid addition salts and esters of the 17β-alkyl-17α-oxy-oestratrienes are also embraced. The addition salts are the corresponding salts with inorganic and organic acids. Suitable addition salts are in particular the hydrochlorides, hydrobromides, acetates, citrates, oxalates, tartrates and methanesulphonates. If R³ and R^{17′} are hydrogen, i.e. a 3,17α-diol is present, the esters of these hydroxyl compounds, too, may be formed. These esters are preferably formed with organic acids, suitable acids being the same acids as for the formulation of the addition salts, i.e. in particular acetic acid, but also higher carboxylic acids, such as, for example, propionic acid, butyric acid, isobutyric acid, valeric acid, isovaleric acid or pivalic acid.

The novel 17β-alkyl-17α-oxy-oestratrienes have a plurality of centres of chirality. Accordingly, in each case there is a plurality of stereoisomeric forms of each compound. The compounds of the formula I may be present as tautomers, stereoisomers or geometrical isomers. The invention furthermore also embraces all possible isomers, such as E and Z isomers, S and R enantiomers, diastereomers, racemates and mixtures of the same including the tautomeric compounds. All these isomeric compounds form—even if not expressly stated—part of the subject matter of the present invention. The isomer mixtures can be separated by customary methods, such as, for example, crystallization, chromatography or salt formation, into the enantiomers or E/Z isomers.

Particularly suitable compounds for the purpose of the invention are oestratrienes having the general formula I, i.e.
11β-Fluoro-17β-methyl-7-[5-[methyl(8,8,9,9,9-pentafluorononyl)amino]pentyl]oestra-1,3,5(10)-triene-3,17α-diol
11β-Fluoro-17β-methyl-7-[5-[(2R)-2-(7,7,8,8,8-pentafluorooctyl)-1-pyrrolidinyl]pentyl]oestra-1,3,5(10)-triene-3,17α-diol
11β-Fluoro-17β-methyl-7-[5-[(2R)-2-(6,6,7,7,7-pentafluoroheptyl)-1-pyrrolidinyl]pentyl]oestra-1,3,5(10)-triene-3,17α-diol
4-Chloro-11β-fluoro-17β-methyl-7-[5-[methyl(8,8,9,9,9-pentafluorononyl)amino]pentyl]oestra-1,3,5(10)-triene-3,17α-diol
4-Bromo-11β-fluoro-17β-methyl-7-[5-[methyl(8,8,9,9,9-pentafluorononyl)amino]pentyl]oestra-1,3,5(10)-triene-3,17α-diol
4-Bromo-11β-fluoro-17β-methyl-7-[5-[(2R)-2-(7,7,8,8,8-pentafluorooctyl)-1-pyrrolidinyl]pentyl]oestra-1,3,5(10)-triene-3,17α-diol
4-Chloro-11β-fluoro-17β-methyl-7-[5-[(2R)-2-(7,7,8,8,8-pentafluorooctyl)-1-pyrrolidinyl]pentyl]oestra-1,3,5(10)-triene-3,17α-diol
4-Bromo-11β-fluoro-17β-methyl-7-[5-[(2R)-2-(6,6,7,7,7-pentafluoroheptyl)-1-pyrrolidinyl]pentyl]oestra-1,3,5(10)-triene-3,17α-diol
4-Chloro-11β-fluoro-17β-methyl-7-[5-[(2R)-2-(6,6,7,7,7-pentafluoroheptyl)-1-pyrrolidinyl]pentyl]oestra-1,3,5(10)-triene-3,17α-diol
2,4-Dichloro-11β-fluoro-17β-methyl-7-[5-[(2R)-2-(7,7,8,8,8-pentafluorooctyl)-1-pyrrolidinyl]pentyl]oestra-1,3,5(10)-triene-3,17α-diol

The 17β-alkyl-17α-oxy-oestratrienes according to the invention also differ from known compounds in that a halogen atom is attached in the 11β-position and an alkyl radical is attached in the 17β-position.

In contrast to 3,17α-dihydroxy-oestratrienes which are unsubstituted in the 17β-position, the 17β-alkyl-17α-oxy-oestratrienes according to the invention form virtually no metabolites. Metabolites may also be biologically active. It has been found that the oestratriene derivatives formed by oxidation of the hydroxyl group attached in the 17α-position, which gives rise to a 17-oxo derivative, have very strong biological activity.

By blocking the 17β-position with an alkyl radical, in particular with a C₁-C₄-alkyl group, this oxidation reaction is forestalled, which also suppresses metabolitic variety. The oestratrienes according to the invention used as active compounds therefore have species-independent efficacy and activity. Accordingly, the advantage of these compounds consists in the fact that the full efficacy of the active compound is realized in a single compound.

For this reason, there are advantages in the development of pharmaceuticals since, owing to the lack of the formation of biologically active metabolites, it is easier to assign the efficacy to certain structural principles, thus allowing a more targeted search for active compounds.

Moreover, the 17β-alkyl-17α-oxy-oestratrienes according to the invention inhibit the action of oestradiol almost by 100%. Accordingly, they are antioestrogens.

To examine the efficacy of the compounds according to the invention, in vivo tests with infant rats were carried out. To this end, the growth of the uterus with peroral administration (p.o.) of the pharmaceutical was examined (test for antioestrogen action).

The principle of this method is to investigate the effect of an administration of compounds having antioestrogen action with simultaneous administration of oestrogens. In rodents, the administration of oestrogens results in an increased weight of the uterus (both by proliferation and by hydropexia). This growth can be inhibited in a dosage-related manner by simultaneous administration of compounds with antioestrogen action.

For the tests, infant female rats having a weight of 35-45 g were examined at the start of the experiment. Five to six animals per dose were tested. For the p.o. administration, the substances were dissolved in one part of ethanol (E) and made up with nine parts of peanut oil (PO). For acclimatization, the young rats just dropped by the mothers were delivered one day before the start of the treatment and immediately supplied with food—right in the cage. For three days, the animals were then treated in combination with 0.5 μg of oestradiol benzoate (EB) once per day. EB was always administrated subcutaneously (s.c.), whereas the test substance was administered p.o. 24 hours after the last administration, the animals were weighed and killed, and the uteri were removed. The moist weights (less contents) of the prepared uteri were determined. The following control studies were carried out: for a negative control, 0.2 ml of an E/PO mixture was administered per animal and day. For a positive control study, 0.5 μg of EB/0.1 ml was administered per animal and day.

For each group, the means with standard deviation (X±SD) and the significance of the differences to the control group (EB) of the relative organ weights (mg/100 g of body weight) were determined in the Dunnett test (p<0.05). The inhibition (in %) relative to the EB control was determined using a calculation programme. The relative efficacy of the test substances was calculated by covariance and regression analysis.

Test results for selective compounds are shown in Table 1. Table 1 shows test results for the uterus growth in rats with simultaneous administration of 0.5 μg of EB/0.1 ml s.c. and peroral administration of the compounds with oestrogen action in an amount in the range of 0.03 mg/kg of body weight.

TABLE 1

| Compound [Ex.] | Inhibition [%] |
|---|---|
| 1 | 42-59 |
| 2 | 70 |
| 3 | 40 |

It can be seen from Table 1 that the antioestrogen action is 50% if a dosage of about 0.03 mg/kg was administered orally.

The compounds according to the invention are as effective or even more effective than the corresponding compounds which are not substituted in the 17β-position. Compared to the compounds not substituted in the 17β-position, the oestratrienes according to the invention also have better compatibility, so that the latter are preferred. The better compatibility can be attributed in particular to the fact that the formation of metabolites is substantially limited.

The compounds according to the invention are furthermore distinguished by extremely high bioavailability, and it is thus possible to achieve high plasma concentrations in the patient in question by administration of the compounds according to the invention. In combination with the high compatibility already mentioned, it is thus possible to carry out a successful and safe therapy because it is possible, using the compounds according to the invention, to establish a serum concentration of active compound which is sufficiently different to the effective concentration of the compound in question. Effective concentration is the minimum active compound plasma concentration required for achieving the desired effect in the respective indication.

The 17β-alkyl-17α-oxy-oestratrienes of the general formula I according to the invention are particularly suitable for preparing pharmaceuticals. Accordingly, the invention also relates to pharmaceutical preparations comprising, in addition to at least one 17β-alkyl-17α-oxy-oestratriene of the general formula I, at least one pharmaceutically compatible carrier.

The pharmaceutical preparations or compositions according to the invention are prepared using customary solid or liquid carriers or diluents and customary pharmaceutical and industrial auxiliaries appropriate for the desired type of administration with a suitable dosage, in a manner known per se. Preferred preparations consist of an administration form suitable for oral, enteral or parenteral, for example i.p. (intraperitoneal), i.v. (intravenous), i.m. (intramuscular) or percutaneous administration. Such administration forms are, for example, tablets, film tablets, coated tablets, pills, capsules, powders, creams, ointments, lotions, liquids, such as syrups, gels, injectable liquids, for example for the i.p., i.v., i.m. or percutaneous injection, etc. In addition, depot forms, such as implant preparations, as well as suppositories, are also suitable. In this case, depending on their type, the individual preparations release to the body the oestratrienes according to the invention gradually or all at once over a short period of time.

For oral administration, it is possible to use capsules, pills, tablets, coated tablets and liquids or other known oral administration forms as pharmaceutical preparations. In this case, the pharmaceuticals can be formulated such that the active compounds are either released over a short period of time and passed onto the body, or have a depot action, so that a longer-lasting, slow supply of active compound to the body is achieved. In addition to the at least one oestratriene, the dosage units may comprise one or more pharmaceutically compatible carriers, for example substances for adjusting the rheology of the pharmaceutical, surfactants, solubilizers, microcapsules, microparticles, granules, diluents, binders, such as starch, sugar, sorbitol and gelatine, furthermore fillers, such as silicic acid and talc, glidants, colorants, perfumes and other substances.

Corresponding tablets can be obtained, for example, by mixing the active compound with known auxiliaries, for example inert diluents such as dextrose, sugar, sorbitol, mannitol, polyvinylpyrrolidone, disintegrants, such as maize starch or alginic acid, binders such as starch or gelatine, glidants, such as carboxypolymethylene, carboxymethylcellulose, cellulose acetate phthalate or polyvinyl acetate. The tablets may also consist of a plurality of layers.

Accordingly, coated tablets can be produced by coating cores that are produced analogously to the tablets with agents that are customary used in coated tablet coatings, for example polyvinylpyrrolidone or Shellac, gum Arabic, talc, titanium oxide or sugar. Here, the shell of the coated tablet may also consist of a plurality of layers, where the auxiliaries that are mentioned above for the tablets may be used.

Capsules that contain active compounds can be produced, for example, by the active compound being mixed with an inert carrier such as lactose or sorbitol and encapsulated in gelatine capsules.

The oestratrienes according to the invention can also be formulated in the form of a solution that is intended for oral administration and that, in addition to the active oestratriene, contains a pharmaceutically compatible oil and/or a pharmaceutically compatible lipophilic surfactant and/or a pharmaceutically compatible hydrophilic surfactant and/or a pharmaceutically compatible water-miscible solvent as components.

To achieve better bioavailability of the active compounds according to the invention, the compounds can also be formulated as cyclodextrin chlatrates. To this end, the compounds are reacted with α-, β- or γ-cyclodextrin or derivatives thereof.

If creams, ointments, lotions and liquids for external application are to be used, they must be constituted such that the compounds according to the invention are supplied to the body in adequate amounts. These administration forms comprise auxiliaries, for example substances for adjusting the rheology of the pharmaceuticals, surfactants, preservatives, solubilizers, diluents, substances for increasing the permeability of the oestratrienes according to the invention through the skin, colorants, perfumes and skin protection agents, such as conditioners and moisturizers. Together with the compounds according to the invention, the pharmaceutical may also comprise other active compounds [Ullmanns Enzyklopädie der technischen Chemie [Ullmann's Encyclopedia of industrial chemistry], Volume 4 (1953), pages 1-39; J. Pharm. Sci., 52, 918 ff. (1963); H. v. Czetsch-Lindenwald, Hilfsstoffe für Pharmazie und angrenzende Gebiete [Auxiliaries for pharmacy and related fields]; Pharm. Ind., 2, 72 ff (1961); Dr. H. P. Fiedler, Lexikon der Hilfsstoffe für Pharmazie, Kosmetik und angrenzende Gebiete [Dictionary of auxiliaries for pharmacy, cosmetics and related fields], Cantor AG, Aulendorf/Württ, 1971].

The substances according to the invention can also be used in suitable solutions, such as, for example, physiological saline, as solution for infusion or injection. For parenteral administration, the active compounds can be dissolved or suspended in a physiologically compatible diluent. Suitable diluents are in particular oily solutions, such as, for example, solutions in sesame oil, castor oil and cottonseed oil. To increase solubility, it is possible to add solubilizers, such as, for example, benzyl benzoate or benzyl alcohol.

To formulate an injectable preparation, any liquid carrier can be used in which the compounds according to the invention are dissolved or emulsified. These liquids frequently also comprise substances for regulating viscosity, surfactants, preservatives, solubilizers, diluents and further additives for making the solution isotonic. Together with the oestratrienes, it is also possible to administer other active compounds.

The oestratrienes according to the invention can also be applied in the form of a depot injection or an implant preparation, for example subcutaneously. Such preparations can be formulated in such a way that a delayed release of active compound is made possible. To this end, known techniques can be used, for example depots that dissolve or operate with a membrane. As inert materials, implants may contain, for example, biodegradable polymers or synthetic silicones, for example silicone gum. For percutaneous administration, the oestratrienes can also be incorporated, for example, into a patch.

It is also possible to incorporate the substances according to the invention into a transdermal system for transdermal administration.

To achieve an improved transdermal skin flow that produces therapeutically effective blood levels, the compounds according to the invention can also be incorporated into transdermal systems as described analogously for other antioestrogens in WO 01/76608. These transdermal systems are distinguished by a specific ratio of 2 penetrants, in particular lauric acid and propylene glycol.

The dosage of the substances of the general formula I according to the invention is determined by the attending physician and depends inter alia on the substance that is administered, on the method of administration, on the disorder that is to be treated and on the severity of the disorder. The amount of the compounds to be administered varies within a wide range and may cover any effective amount. Depending on the condition to be treated and on the type of administration, the amount of the compound administered may be 0.02-20 mg/kg of bodyweight, preferably 0.1-1 mg/kg of bodyweight, per day. In humans, this corresponds to a daily dose of 1-1000 mg. The preferred daily dosage in humans is 5-50 mg. This applies in particular to tumour therapy. The dose can be given as a single dose to be administered once or divided into two or more daily doses.

The compounds of the general formula I are compounds with very strong antioestrogen activity.

The compounds are suitable for the therapy of oestrogen-dependent disorders, for example breast cancer (second-line therapy of tamoxifen-resistant breast cancer; for adjuvant treatment of breast cancer instead of tamoxifen), endometrial carcinoma, ovarial carcinoma, prostate hyperplasia, anovulatory infertility and melanoma.

The compounds of the general formula I can be used together with antigestagens (competitive progesterone antagonists) for the treatment of hormone-dependent tumours (EP 310 542 A).

The compounds of the general formula I can also be combined together with other active compounds having antiproliferative action. Preference is given to combinations with aromatase inhibitors such as anastrozole, letrozole or exemestane.

Other indications for which the compounds of the general formula I can be used are male hair loss, diffuse alopecia, alopecia caused by chemotherapy and also hirsutism (Hye-Sun Oh and Robert C. Smart, Proc. Natl. Acad. Sci. USA, 93 (1996) 12525-12530).

Moreover, the compounds of the general formula I can be used for preparing medicaments for treating gynaecological disorders, such as, for example, endometriosis.

For gynaecological disorders, the compounds according to the invention can also be combined with gestagen and/or oestrogens. The compounds of the general formula I can be used, for example, as a component in the products described inter alia in EP 346 014 B1, which products comprise an oestrogen and a pure antioestrogen and are intended for the simultaneous, sequential or separate use for the selective oestrogen therapy of peri- or postmenopausal women.

The compounds of the general formula I can furthermore be used for preparing pharmaceutical compositions for male and female fertility control (male fertility control: DE 195 10 862.0 A).

The oestratrienes according to the invention can be prepared analogously to known processes:

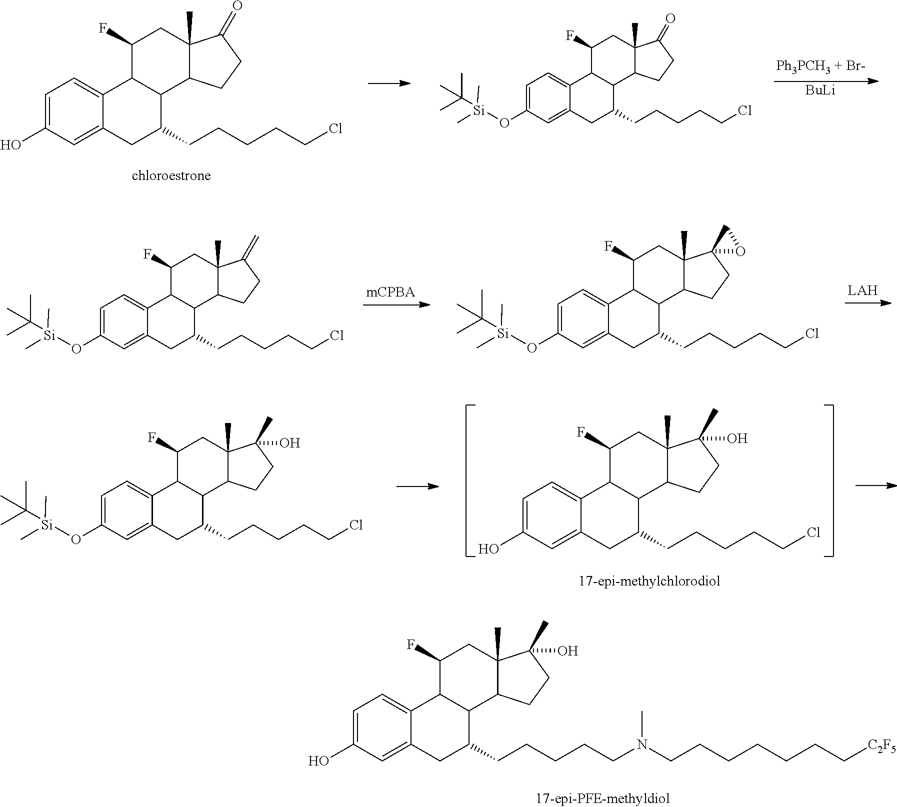

Scheme 1 shows a reaction scheme which allows the preparation of the compounds according to the invention.

In principle, all of the compounds listed can be prepared from the 17-oxo compound. The preparation of the 17-oxo compounds is described in an exemplary manner for instance in WO 99/33855 [CAS: 204138-84-1 (Cl-on, 2b), CAS: 204138-92-1 (Br-on, 1 g), CAS: 204138-93-2 (Br-ol)]. Other derivatives than the compounds expressly disclosed in this document having the same substitution pattern can be prepared analogously. In the same manner, it is also possible to prepare the oestratrienes according to the invention from the 17α-hydroxy or 17α-alkoxy compounds (“17α-OH”). The preparation of these derivatives is likewise described, for example, in WO 99/33855. In the same manner, this document also discloses the preparation of the 17α-hydroxy or 17α-alkoxy compounds and the 17-oxo compounds having an amine grouping in the side chain in the 7α-position. If the preparation of the starting materials has not been described, the starting materials are known or commercially available, or the compounds are synthesized analogously to the processes described. The preparation of some precursors, intermediates and products is described in an exemplary manner below.

For preparing the substances according to the invention, use is made, for example, of the following processes (see also EP 0138 504 B1; WO 97/45441 A1; WO 98/07740 A1; WO 99/33855 A1):

The side chain in the 7α-position can be constructed, for example, by the procedure given in WO 98/07740 A1.

In the process steps described below, the radicals R¹, R², R³, R⁴, R⁵, R⁶, Hal, R^{17′}, R^{17″}, U, V, X, Y, E have the meanings given above.

PG represents a protective group, for example ethers, esters or carbonates. Particularly suitable are alkyl or silyl ethers, with butyldimethylsilyl and tetrahydropyranyl ether being preferred.

LG represents a leaving group, for example a sulphonate group or halogen. Tosylate or mesylate groups, and also chlorine, bromine or iodine, are particularly suitable. Preference is given to chlorine or a mesylate group.

R²⁰ represents hydrogen or an optionally mono- or polyfluorinated C₁-C₃-alkyl, C₂-C₃-alkenyl or C₂-C₃-alkynyl radical.

In the optional process step a), the 3-hydroxyl group of an 11-halo-7-chain-substituted 3-hydroxyoestr-17-one of the general formula III (WO 99/33855) is, by reaction with a silylating agent or dihydropyran, converted, for example, into a silyl ether- or tetrahydropyranyl ether-protected compound of the general formula IV. The reaction is carried out in an aprotic solvent, for example dichloromethane, in the presence of a base, for example imidazole, or an acid, for example pyridinium para-toluenesulphonic acid, at room temperature (0-100° C.) over a period of 3 hours (1 to 24 hours).

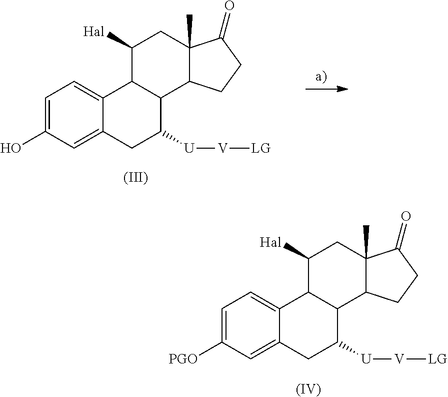

Alternatively, process step a) is carried out according to (D. W. Hansen and D. Pilipauskas J. Org. Chem. (1985) 945 or K. F. Bernady et al. J. Org. Chem. (1979) 1438).

In a Wittig reaction, the protected or free 7α-substituted oestrone derivative of the general formula (IV) can be converted using, for example, a methyltriphenylphosphonium halide in an aprotic solvent, for example tetrahydrofuran, dimethyl sulphoxide or toluene, in the presence of a base, for example butyllithium, at room temperature (20-100° C.), over a period of 8 hours (1 to 24 hours), into the 17-exomethylene steroid of the general formula (V) (process step b)).

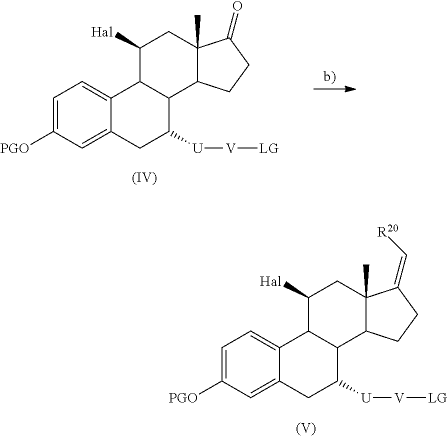

In step c), the exocyclic olefin of the general formula (V) can be converted into the 17-spiro epoxide of the general formula (VI) using, for example, meta-chloroperbenzoic acid in an aprotic solvent, for example dichloromethane, at room temperature (0-100° C.) over a period of 4 hours (1-18 hours). The crude product (VI) of this reaction is not storage-stable and can immediately, without purification, be converted in step d) using, for example, lithium aluminium hydride in an aprotic solvent, for example tetrahydrofuran or toluene, at 10° C. (0-80° C.) over a period of 2 hours (1-24 hours) into the 17β-alkyl-oestra-3,17α-diol of the general formula (VII), where the group —CH₂—R²⁰ in position 17 represents the radical R^{17″}.

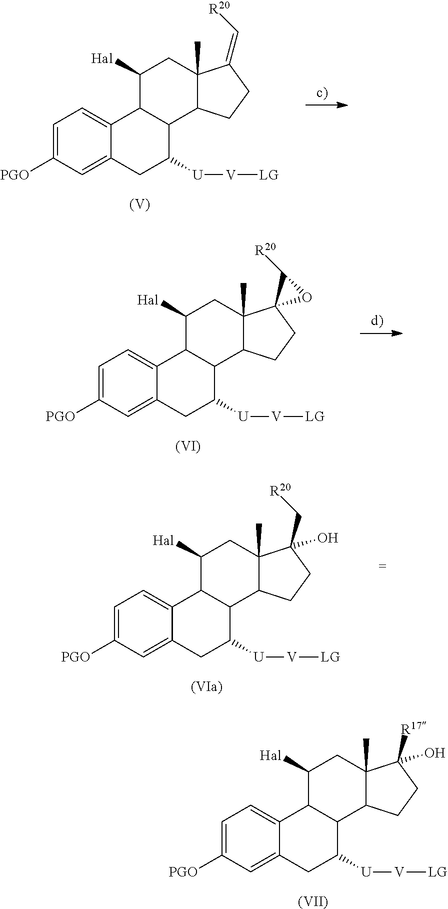

In step e), the protected or free 17β-alkyl-oestra-3,17α-diol of the general formula (VII) can optionally be converted into the halogenated oestradiol of the general formula (VIII), for example by adding a halogenating agent, for example 2, 3,4,5,6,6-hexachloro-2,4-cyclohexadien-1-one, in a polar aprotic solvent, for example dimethylformamide, at room temperature (0-100° C.) over a period of 22 hours (4-48 hours).

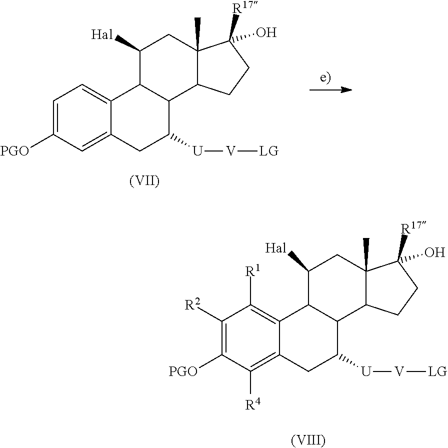

In step f), the protected or free halogenated oestradiol of the general formula (VIII) can, prior to any removal of protective groups in the 3-position with an organic acid, for example oxalic acid, or fluoride, for example tetrabutylammonium fluoride, in a polar solvent, for example water or ethanol, at room temperature (0-100° C.) over a period of 12 hours (4-48 hours), optionally be converted into an oestradiol derivative of the general formula (IX), for example by addition of an acylating or etherifying agent, for example an acyl anhydride, an acyl halide or an alkyl halide, in a polar aprotic solvent, for example pyridine or dimethylformamide, at room temperature (0-100° C.), over a period of 12 hours (4-48 hours).

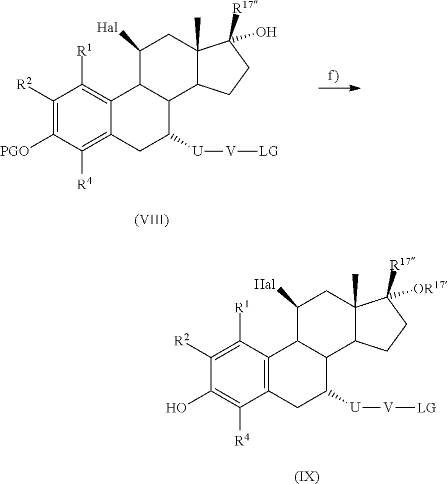

In a step g), the oestradiol derivative of the general formula (IX) can be converted with an amine building block of the general formula (XV) in a polar aprotic solvent, for example dimethylformamide, if appropriate in the presence of an inorganic base, for example sodium carbonate, and an iodide salt, for example sodium iodide, at room temperature (20-100° C.) over a period of 8 hours (4-48 hours) into the amine of the general formula (I).

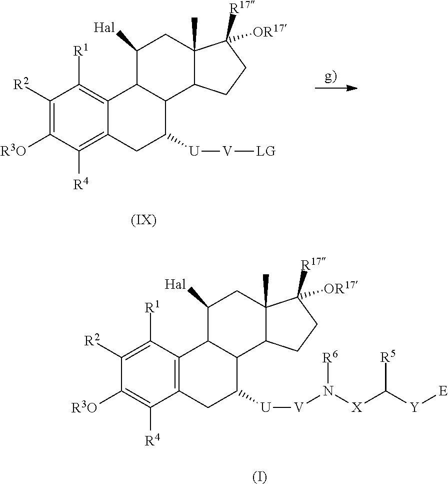

### General Preparation of an Amine Building Block of the General Formula (XV):

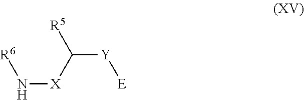

### Iodination:

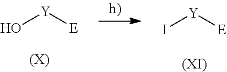

In a step h), an alcohol of the general formula (X) can be converted, for example by dropwise addition to a reaction solution of triphenylphosphine and iodine in dichloromethane at room temperature (20-100° C.) over a period of 1 hour (0.25-8 hours), into the iodide of the general formula (XI).

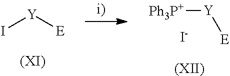

In a step i), the Wittig salt of the general formula (XII) can be obtained by adding the iodide of the general formula (XI) to a suspension of triphenylphosphine in an aprotic polar solvent, for example acetonitrile, and stirring at boiling point (40-100° C.) for a period of 8 hours (2-24 hours).

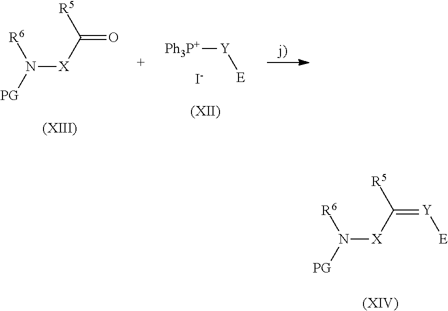

In a step j), an aldehyde or ketone of the general formula (XIII) can be converted at −40° C. (−78-30° C.) into an optionally protected olefin of the general formula (XIV) by dropwise addition to a reaction solution of a Wittig salt of the general formula (XII) and a base, for example sodium hexamethyldisilazane, in an aprotic solvent, for example tetrahydrofuran, warming to room temperature (0-80° C.) and stirring at this temperature for a further hour (0.5-10 hours).

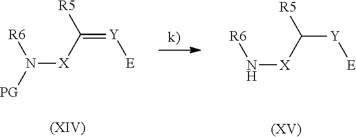

In a step k), an optionally protected olefin of the general formula (XIV) can be hydrogenated in a polar solvent, for example aqueous ethanol, with hydrogen under atmospheric pressure (1-20 bar) in the presence of a hydrogenation catalyst, for example platinum oxide, at room temperature (0-80° C.) for a period of 1 hour (0.5-8 hours) and, if appropriate, be deprotected to the amine of the general formula (XV), for example using an acid, for example hydrochloric acid.

### Preparation of an Alcohol of the General Formula (X):

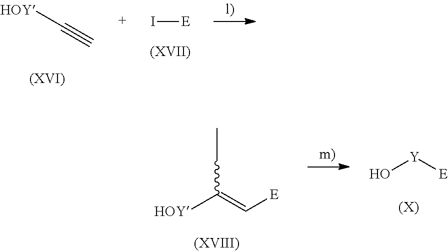

An alkynol of the general formula (XVI) (where —Y′(CH₂)₂— represents —Y—) can be converted, for example in a step I), in a polar aprotic solvent, for example acetonitrile, in the presence of sodium bicarbonate and sodium dithionate at −10° C. (−30-30° C.) with an iodide of the general formula (XVII) into the vinyl iodide of the general formula (XVIII).

In a step m), the vinyl iodide of the general formula (XVIII) can be hydrogenated with hydrogen under atmospheric pressure (1-20 bar) in the presence of a hydrogenation catalyst, for example palladium on activated carbon, at room temperature (0-80° C.) over a period of 1 hour (0.5-8 hours) into the alcohol of the general formula (X).

### A. PREPARATION OF THE COMPOUNDS ACCORDING TO THE INVENTION

### Example 1

### [5-(11β-Fluoro-3,17α-dihydroxy-17β-methyloestra-1,3,5(10)-trien-7α-yl)pentyl](methyl)(8,8,9,9,9-pentafluorononyl)amine

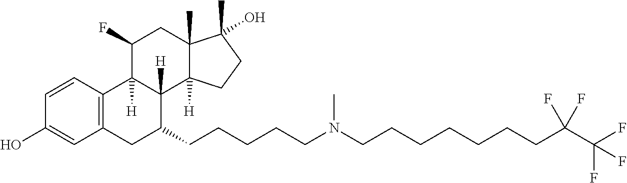

### 1.1 Preparation of the Intermediates

### 1.1.1 3-(tert-Butyldimethylsilyloxy)-7α-(5-chloropentyl)-11β-fluorooestra-1,3,5(10)-trien-17-one

At room temperature, 3.81 g of imidazole were added to a solution of 15.7 g of 7α-(5-chloropentyl)-11β-fluoro-3-hydroxy-17β-methyloestra-1,3,5(10)-trien-17-one [WO 99/33855, CAS: 204138-84-1] in 160 ml of dichloromethane, the mixture was cooled to 10° C., 8.44 g of tert-butyldimethylsilyl chloride were added a little at a time and the mixture was stirred at room temperature for 3 hours. For work-up, a saturated sodium bicarbonate solution (40 ml) and 80 ml of water were added with ice-cooling, the phases were separated and the organic phase was dried over sodium sulphate and concentrated under reduced pressure. This gave 21 g of crude 3-(tert-butyldimethylsilyloxy)-7α-(5-chloropentyl)-11β-fluorooestra-1,3,5(10)-trien-17-one.

H-NMR: 400 MHz, CDCl3, δ=7.16 (d, 1H), 6.67 (d (br), 1H), 6.58 (s (br), 1H), 5.58 (d, 1H), 3.51 (t, 2H), 0.98 (s, 9H), 0.91 (s, 3H), 0.19 (s, 6H).

### 1.1.2 3-(tert-Butyldimethylsilyloxy)-7α-(5-chloropentyl)-11β-fluoro-17-methyleneoestra-1,3,5(10)-triene

At room temperature, 36 ml of a butyllithium solution (2.5M in hexane) were added over a period of 10 minutes to a solution of 28.58 g of methyltriphenylphosphonium bromide in 500 ml of tetrahydrofuran, the mixture was stirred at room temperature for 20 minutes and then warmed to 70° C., a solution of 10.14 g of 3-(tert-butyldimethylsilyloxy)-7α-(5-chloropentyl)-11β-fluorooestra-1,3,5(10)-trien-17-one in 280 ml of tetrahydrofuran was added dropwise over a period of 80 minutes and the mixture was stirred at a bath temperature of 95° C. for 2 hours. For work-up, 160 ml of water were added dropwise with ice-cooling, the phases were separated, the aqueous phase was extracted with ethyl acetate and the combined organic phases were washed with saturated sodium chloride solution, dried over sodium sulphate and concentrated under reduced pressure.

This gave 27 g of a first crude product. Four times, this was stirred with 100 ml of hexane at 40° C., decanted and dried under reduced pressure. This gave 11.6 g of purified crude product 3-(tert-butyldimethylsilyloxy)-7α-(5-chloropentyl)-11β-fluoro-17-methyleneoestra-1,3,5(10)-triene.

H-NMR: 300 MHz, CDCl3, δ=7.18 (d, 1H), 6.67 (m, 1H), 6.57 (s (br), 1H), 5.57 (d (br), 1H), 4.67 (m, 2H), 3.51 (t, 2H), 0.97 (s, 9H), 0.19 (s, 6H).

### 1.1.3 3-(tert-Butyldimethylsilyloxy)-7α-(5-chloropentyl)-11β-fluoro-(17R)-spiro-[oestra-1,3,5(10)-triene-17,2′-oxirane]

At room temperature, 3.05 g of m-chloroperbenzoic acid were added a little at a time to a solution of 5.32 g of 3-(tert-butyldimethylsilyloxy)-7α-(5-chloropentyl)-11β-fluoro-17-methyleneoestra-1,3,5(10)-triene in 55 ml of dichloromethane, and the mixture was stirred for 4 hours. For work-up, 50 ml of saturated sodium bicarbonate solution and 50 ml of methyl tert-butyl ether were added, the phases were separated, the aqueous phase was extracted with methyl tert-butyl ether and the combined organic phases were dried over sodium sulphate and concentrated under reduced pressure. This gave 5.6 g of crude 3-(tert-butyldimethylsilyloxy)-7α-(5-chloropentyl)-11β-fluoro-(17R)-spiroqoestra-1,3,5(10)-triene-17,2′-oxirane].

H-NMR: 300 MHz, CDCl3, δ=7.16 (d, 1H), 6.67 (m, 1H), 6.57 (s (br), 1H), 5.57 (d, 1H), 3.51 (t, 2H), 3.22 (s, 1H), 1.02 (s (br), 3H), 0.98 (s, 12H), 0.19 (s, 6H).

### 1.1.4 3-(tert-Butyldimethylsilyloxy)-7α-(5-chloropentyl)-11β-fluoro-17β-methyloestra-1,3,5(10)-trien-17α-ol

At 10° C., a second solution of 5.27 g of 3-(tert-butyldimethylsilyloxy)-7α-(5-chloropentyl)-11β-fluoro-(17R)-spiro-[oestra-1,3,5(10)-triene-17,2′-oxirane] in 20 ml of tetrahydrofuran was added dropwise to a 2M solution of lithium aluminium hydride (5.03 ml), and the mixture was stirred at room temperature for 2 hours. For work-up, 8 ml of acetone and 70 ml of citric acid solution were added, the mixture was diluted with 80 ml of ethyl acetate, the phases were separated, the aqueous phase was extracted with ethyl acetate and the combined organic phases were washed with sodium chloride solution, dried over sodium sulphate, concentrated under reduced pressure and chromatographed on silica gel using hexane/ethyl acetate. This gave 2.75 g of pure 3-(tert-butyldimethylsilyloxy)-7α-(5-chloropentyl)-11β-fluoro-17β-methyloestra-1,3,5(10)-trien-17α-ol.

H-NMR: 300 MHz, CDCl3, δ=7.17 (d, 1H), 6.65 (m, 1H), 6.57 (s (br), 1H), 5.52 (d, 1H), 3.51 (t, 2H), 1.26 (d, 3H), 0.98 (s, 9H), 0.91 (d, 3H), 0.19 (s, 6H).

### 1.1.5 Methyl-(8,8,9,9,9-pentafluorononyl)amine

At −20° C., 4.0 g of methylamine were condensed into a solution of 2.9 g of 8,8,9,9,9-pentafluorononyl tosylate [WO 99/33855, page 20, CAS: 228570-38-5] in 10 ml of abs. tetrandyrofuran, and the mixture was stirred in a pressure container at room temperature overnight. The pressure container was vented at −20° C. and allowed to warm to room temperature to let excess methylamine evaporate. The reaction solution was taken up in dichloromethane, washed with water, dried over magnesium sulphate and concentrated under reduced pressure. This gave 1.58 g of methyl-(8,8,9,9,9-pentafluorononyl)amine as a crude product.

H-NMR: 300 MHz, CDCl3, δ=2.60 (t, 2H), 2.47 (s, 3H), 1.94-2.14 (m, 2H), 1.57-1.68 (d, 2H), 1.48-1.56 (m, 2H), 1.34-1.46 (m, 6H).

### 1.2 Preparation of the End Product

0.22 g of sodium carbonate, 0.63 g of sodium iodide and 0.78 g of (8,8,9,9,9-pentafluoro-nonyl)methylamine were added to a solution of 1.1 g of 3-(tert-butyldimethylsilyloxy)-7α-(5-chloropentyl)-11β-fluoro-17β-methyloestra-1,3,5(10)-trien-17α-ol in 6.29 ml of dimethylformamide, and the mixture was stirred at 80° C. for 5 hours. For work-up, the mixture was diluted with 10 ml of ethyl acetate and washed with water and sodium bicarbonate solution, and the organic phase was dried over magnesium sulphate, filtered and concentrated under reduced pressure. This crude product was dissolved in 9.44 ml of ethanol, 0.73 g of potassium fluoride and 0.2 ml of water were added and the mixture was stirred at room temperature for 14 hours. The mixture was then concentrated under reduced pressure and chromatographed on silica gel using hexane/ethyl acetate. This gave 577 mg of pure [5-(11β-fluoro-3,17α-dihydroxy-17β-methyloestra-1,3,5(10)-trien-7α-yl)pentyl](methyl)(8,8,9,9,9-pentafluorononyl)amine.

H-NMR: 600 MHz, CDCl3, δ=7.18 (d, 1H), 6.63 (m, 1H), 6.52 (s (br), 1H), 5.60 (d, 1H), 2.21 (s, 3H), 1.26 (s, 3H), 0.88 (d, 3H).

### Example 2

### 11β-Fluoro-17β-methyl-7α-[5-[(2R)-2-(7,7,8,8,8-pentafluorooctyl)-1-pyrrolidinyl]pentyl]-oestra-1,3,5(10)-triene-3,17α-diol

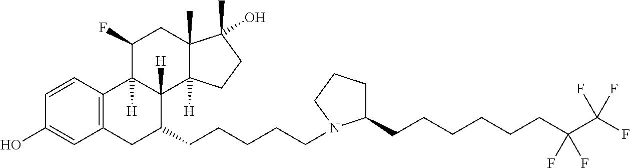

### 2.1 Preparation of the Intermediates

### 2.1.1 (R)-2-(7,7,8,8,8-Pentafluorooctyl)pyrrolidine

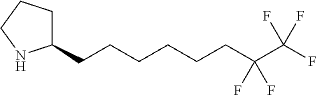

### a) 6,6,7,7,7-Pentafluoro-4-iodohept-4-en-1-ol

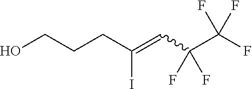

At −12° C., 507 g of pentafluoroiodoethane were added to a solution of 150 g of 4-pentyn-1-ol in 1.5 l of acetonitrile and 1.14 l of water. A mixture of 150 g of sodium bicarbonate and 294 g of sodium dithionite (85% pure) was then added, and the mixture was stirred at from −10 to 0° C. for 1 hour. The reaction was checked by TLC and the reaction mixture was then added to water and extracted with ethyl acetate, and the extract was washed with saturated sodium chloride solution and dried over sodium sulphate. After filtration, the extract was concentrated carefully on a rotary evaporator at 100 mbar. This gave 6,6,7,7,7-pentafluoro-4-iodohept-4-en-1-ol as a crude product (650 g).

### b) 6,6,7,7,7-Pentafluoroheptan-1-ol CAS [344452-11-5]

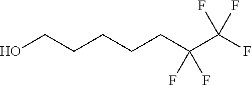

At room temperature, 118 g of palladium-1-carbon were added to a solution of 650 g of crude 6,6,7,7,7-pentafluoro-4-iodohept-4-en-1-ol in 3 l of ethyl acetate and 500 ml of triethylamine, and the mixture was then hydrogenated under a hydrogen atmosphere at atmospheric pressure, and the progress of the reaction was monitored by 1H-NMR. After filtration and washing of the catalyst with ethyl acetate, the organic phase was washed with water, 1% strength hydrochloric acid and saturated sodium chloride solution. After drying with sodium sulphate and careful concentration of the solvent down to 50 mbar, the residue was distilled under oil pump vacuum (0.5 mbar) at 65° C. to give 6,6,7,7,7-pentafluoroheptan-1-ol (235 g).

H-NMR: 400 MHz, CDCl₃, δ=3.66 (t, 2H), 2.03 (m, 2H), 1.40-1.70 (m, 7H).

### c) 1,1,1,2,2-Pentafluoro-7-iodoheptane

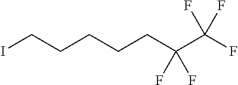

At room temperature, 61.56 g of iodine were added with cooling to a solution of 64.25 g of triphenylphosphine in 500 ml of dichloromethane, and the mixture was stirred for 15 minutes. Over a period of 1 hour, a solution of 50 g of 6,6,7,7,7-pentafluoroheptan-1-ol in 60 ml of dichloromethane was then added dropwise, and the mixture was stirred for another hour. The mixture was added to water and extracted with dichloromethane, and the organic phase was washed with saturated sodium chloride solution, dried over sodium sulphate, filtered off and carefully concentrated on a rotary evaporator. The residue was digested with 300 ml of hexane and the hexane phase obtained was distilled at 10 mbar, which gave 1,1,1,2,2-pentafluoro-7-iodoheptane (62.4 g).

H-NMR: 300 MHz, CDCl₃, δ=3.20 (t, 2H), 2.03 (m, 2H), 1.86 (m, 2H), 1.43-1.69 (m, 4H).

### d) (6,6,7,7,7-Pentafluoroheptyl)triphenylphosphonium iodide

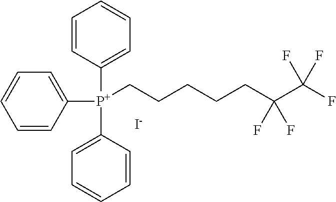

313 g of 1,1,1,2,2-pentafluoro-7-iodoheptane were added to a suspension of 260 g of triphenylphosphine in 1.26 l of acetonitrile, and the mixture was heated under reflux for 18 hours. The solvent was concentrated, and the product was recrystallized twice from tert-butyl methyl ether. This gave (6,6,7,7,7-pentafluoroheptyl)triphenylphosphonium iodide (567 g) as a white powder.

H-NMR: 300 MHz, CDCl₃, δ=7.66-7.91 (m, 15H), 3.76 (m, 2H), 1.50-2.10 (m, 8H).

### e) tert-Butyl (S)-2-[(Z)-7,7,8,8,8-pentafluorooct-1-enyl]pyrrolidine-1-carboxylate

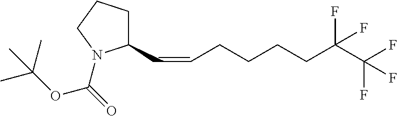

At −40° C., 738 ml of sodium hexamethyldisilazane (1M in tetrahydrofuran) were added over a period of 10 minutes to a solution of 468.38 g of (6,6,7,7,7-pentafluoroheptyl)triphenylphosphonium iodide in 3.8 l of tetrahydrofuran, and the mixture was left at this temperature for 2 hours. Over a period of 15 minutes, a solution of 147 g of tert-butyl (2S)-2-formyl-1-pyrrolidinecarboxylate in 850 ml was then added, and the mixture was stirred at −40° C. for 1 hour, warmed to room temperature and stirred for another hour. After addition of 2.5 l of hexane, the mixture was concentrated to ⅓ of its original volume, tert-butyl methyl ether was added and the resulting precipitate was allowed to settle. The mixture was then filtered, the filtercake was washed with butyl methyl ether and the filtrate was concentrated to dryness. Flash-chromatography on silica gel (hexane/ethyl acetate) gave tert-butyl (S)-2-[(Z)-7,7,8,8,8-pentafluorooct-1-enyl]pyrrolidine-1-carboxylate (155.7 g).

H-NMR: 300 MHz, CDCl₃, δ=5.33 (m, 2H), 4.47 (br. s, 1H), 3.37 (m, 2H), 1.41 (s, 9H).

### f) tert-Butyl (R)-2-(7,7,8,8,8-pentafluorooctyl)pyrrolidine-1-carboxylate

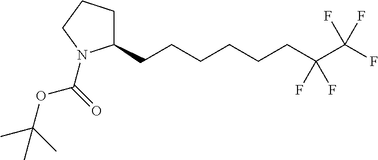

At room temperature, 6.19 g of platinum oxide were added to a solution of 61.9 g of tert-butyl (S)-2-(7,7,8,8,8-pentafluorooct-1-enyl)pyrrolidine-1-carboxylate in 1.4 l of methanol and 300 ml of water, and the mixture was hydrogenated under atmospheric pressure until the hydrogen uptake had been completed. After filtration through a layer of Celite, the filtrate was concentrated, taken up in ethyl acetate, the aqueous phase was separated off and the organic phase was dried over sodium sulphate and concentrated. Filtration of the solution through a PTFE filter was followed by evaporation to dryness. The resulting tert-butyl (R)-2-(7,7,8,8,8-pentafluorooctyl)pyrrolidine-1-carboxylate (59.6 g) was used directly, as a crude product, for the next step.

H-NMR: 300 MHz, CDCl₃, δ=3.71 (br. s, 1H), 3.21-3.43 (m, 2H), 1.44 (s, 9H).

### g) (R)-2-(7,7,8,8,8-Pentafluorooctyl)pyrrolidine

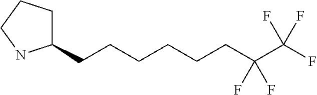

At room temperature, 35 ml of hydrochloric acid (37% strength) were added to a solution of 35 g of tert-butyl (R)-2-(7,7,8,8,8-pentafluorooctyl)pyrrolidine-1-carboxylate in 636 ml of 1,4-dioxane, and the mixture was then heated at 50° C. for 1 hour. The mixture was then concentrated, methylene chloride was added, the mixture was washed 2× with saturated sodium bicarbonate solution and water, dried over magnesium sulphate and concentrated and the product was purified by kugelrohr distillation (bp. 110-125° C., 1 torr). This gave (R)-2-(7,7,8,8,8-pentafluorooctyl)pyrrolidine (21.21 g).

H-NMR: 400 MHz, CDCl₃, δ=3.00 (ddd, 1H), 2.92 (m, 1H), 2.81 (ddd, 1H), 2.00 (m, 2H), 1.87 (m, 1H), 1.52-1.80 (m, 6H), 1.28-1.51 (m, 8H), 1.21 (m, 1H).

### 2.2 Preparation of the End Product

Analogously to step 1.2 of Example 1, 1.1 g of 3-(tert-butyldimethylsilyloxy)-7α-(5-chloropentyl)-11β-fluoro-17β-methyloestra-1,3,5(10)-triene-3,17α-diol and 860 mg of (R)-2-(7,7,8,8,8-pentafluorooctyl)pyrrolidine as amine gave, after flash chromatography, 116-fluoro-17β-methyl-7α-[5-[(2R)-2-(7,7,8,8,8-pentafluorooctyl)-1-pyrrolidinyl]pentyl]oestra-1,3,5(10)-triene-3,17α-diol (772 mg).

H-NMR: 400 MHz, CDCl₃, δ=7.19 (d, 1H), 6.65 (m, 1H), 6.55 (s (br), 1H), 5.60 (d, 1H), 3.14 (m, 1H), 2.91 (m, 1H), 2.54 (m, 1H), 1.26 (s, 3H), 0.88 (d, 3H).

### Example 3

### 11β-Fluoro-17β-methyl-7α-[5-[(2R)-2-(6,6,7,7,7-pentafluoroheptyl)-1-pyrrolidinyl]pentyl]oestra-1,3,5(10)-triene-3,17α-diol

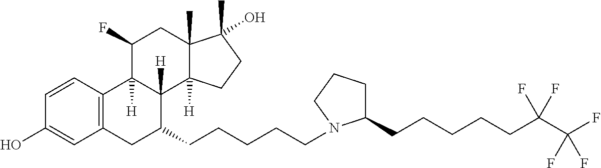

### 3.1 Preparation of the Intermediates

### 3.1.1 (R)-2-(6,6,7,7,7-Pentafluoroheptyl)pyrrolidine

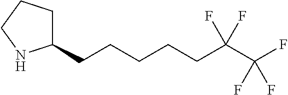

### a) 5,5,6,6,6-Pentafluoro-3-iodohex-3-en-1-ol

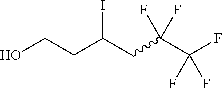

Analogously to the method described in Example 2.1.1 a) 280 g of 3-butyn-1-ol and 1136 g of pentafluoroethyl iodide gave 5,5,6,6,6-pentafluoro-3-iodohex-3-en-1-ol (1205 g) as a crude product which was used directly for the next step.

### b) 5,5,6,6,6-Pentafluorohexan-1-ol (ZK 6074973, CAS [58556-45-9])

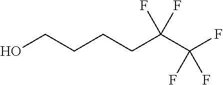

Analogously to the method described in Example 2.1.1 b), 319.5 g of 5,5,6,6,6-pentafluoro-3-iodohex-3-en-1-ol gave 5,5,6,6,6-pentafluorohexan-1-ol (223 g).

H-NMR: 300 MHz, CDCl₃, δ=3.69 (t, 2H), 2.06 (m, 2H), 1.54-1.77 (m, 5H).

### c) 1,1,1,2,2-Pentafluoro-6-iodohexane

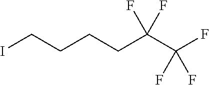

Analogously to the method described in Example 2.1.1 c) 223 g of 5,5,6,6,6-pentafluoro-hexan-1-ol gave 1,1,1,2,2-pentafluoro-6-iodohexane (223 g).

H-NMR: 300 MHz, CDCl₃, δ=3.20 (t, 2H), 1.85-2.15 (m, 4H), 1.66-1.79 (m, 2H).

### d) (5,5,6,6,6-Pentafluorohexyl)triphenylphosphonium iodide

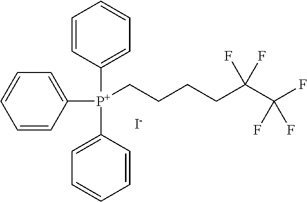

Analogously to the method described in Example 2.1.1 d), 334 g of 1,1,1,2,2-pentafluoro-6-iodohexane gave (5,5,6,6,6-pentafluorohexyl)triphenylphosphonium iodide (671 g).

H-NMR: 300 MHz, CDCl₃, δ=7.65-7.92 (m, 15H), 3.88 (m, 2H), 1.73-2.22 (m, 6H).

### e) tert-Butyl (S)-2-[(Z)-6,6,7,7,7-pentafluorohept-1-enyl]pyrrolidine-1-carboxylate

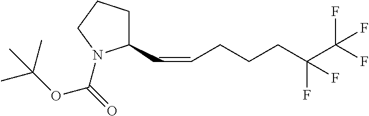

Analogously to the method described in Example 2.1.1 e), 318 g of (5,5,6,6,6-pentafluorohexyl)triphenylphosphonium iodide (671 g) gave tert-butyl (S)-2-[(Z)-6,6,7,7,7-pentafluorohept-1-enyl]pyrrolidine-1-carboxylate (132.7 g).

H-NMR: 300 MHz, CDCl₃, δ=5.29-5.49 (m, 2H), 4.52 (m, 1H), 3.44 (m, 2H), 2.28 (m, 2H), 1.46 (s, 9H).

### f) tert-Butyl (R)-2-(6,6,7,7,7-pentafluoroheptyl)pyrrolidine-1-carboxylate

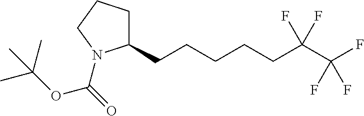

Analogously to the method described in Example 2.1.1 f), 132 g of tert-butyl (S)-2-((Z)-6,6,7,7,7-pentafluorohept-1-enyl)pyrrolidine-1-carboxylate gave tert-butyl (R)-2-(6,6,7,7,7-pentafluoroheptyl)pyrrolidine-1-carboxylate (135 g).

H-NMR: 300 MHz, CDCl₃, δ=3.72 (br. s, 1H), 3.23-3.39 (m, 2H), 1.44 (s, 9H).

### g) (R)-2-(6,6,7,7,7-Pentafluoroheptyl)pyrrolidine

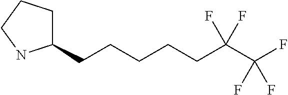

Analogously to the method described in example 2.1.1 g), 20.5 g of tert-butyl (R)-2-(6,6,7,7,7-pentafluoroheptyl)pyrrolidine-1-carboxylate gave (R)-2-(6,6,7,7,7-pentafluoroheptyl)pyrrolidine (13 g).

H-NMR: 400 MHz, CDCl₃, δ=4.09 (br s, 1H), 3.00 (m, 2H), 1.61-1.94 (m, 5H), 1.21-1.59 (m, 9H).

### 3.2 Preparation of the End Product

Analogously to step 1.2 of Example 1, 1.1 g of 3-(tert-butyldimethylsilyloxy)-7α-(5-chloropentyl)-11β-fluoro-17β-methyloestra-1,3,5(10)-triene-3,17α-diol and 820 mg of (R)-2-(7,7,8,8,8-pentafluoroheptyl)pyrrolidine as amine gave, after flash chromatography, 118-fluoro-17β-methyl-7α-[5-[(2R)-2-(6,6,7,7,7-pentafluoroheptyl)-1-pyrrolidinyl]pentyl]oestra-1,3,5(10)-triene-3,17α-diol (760 mg).

H-NMR: 400 MHz, CDCl₃, δ=7.18 (d, 1H), 6.63 (m, 1H), 6.53 (s (br), 1H), 5.60 (d, 1H), 3.16 (m, 1H), 2.90 (m, 1H), 2.54 (m, 1H), 1.26 (s, 3H), 0.88 (d, 3H).

### Example 4

### 4-Chloro-11β-fluoro-17β-methyl-7α-[5-[methyl(8,8,9,9,9-pentafluorononyl)amino]pentyl]-oestra-1,3,5(10)-triene-3,17α-diol

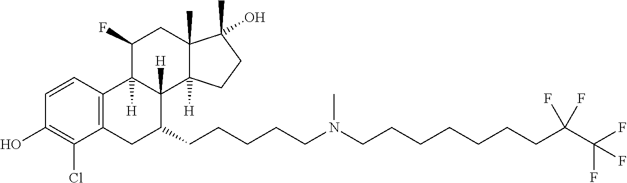

### 4.1 Preparation of the Intermediates

### 4.1.1 3-(tert-Butyldimethylsilyloxy)-4-chloro-7α-(5-chloropentyl)-11β-fluoro-17β-methyloestra-1,3,5(10)-trien-17α-ol

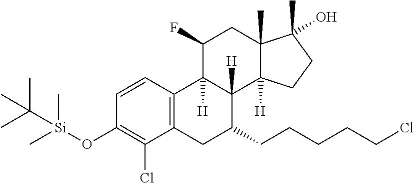

Under an atmosphere of nitrogen, 505 mg of 2,3,4,5,6,6-hexachloro-2,4-cyclohexadien-1-one were added to a solution of 732 mg of 3-(tert-butyldimethylsilyloxy)-7α-(5-chloropentyl)-11β-fluoro-17β-methyloestra-1,3,5(10)-trien-17α-ol in 9 ml of dimethylformamide, and the mixture was stirred at room temperature for 22 hours. This was followed by a further addition of 126 mg of 2,3,4,5,6,6-hexachloro-2,4-cyclohexadien-1-one, and a subsequent last 126 mg-portion after 7 hours. The reaction mixture was stirred for a further 56 hours and then concentrated, and the crude product was chromatographed repeatedly on silica gel (hexane/ethyl acetate). This gave 444 mg of 3-(tert-butyldimethylsilyloxy)-4-chloro-7α-(5-chloropentyl)-11β-fluoro-17β-methyloestra-1,3,5(10)-trien-17α-ol.

H-NMR: 400 MHz, CDCl₃, δ=7.12 (d, 1H), 6.75 (m, 1H), 5.59 (d, 1H), 3.51 (t, 2H), 3.06 (d, 1H), 1.26 (s, 3H), 1.03 (s, 9H), 0.87 (d, 3H), 0.23 (s, 3H), 0.22 (s, 3H).

### 4.2 Preparation of the End Product

Analogously to step 1.2 of Example 1, 428 mg of 3-(tert-butyldimethylsilyloxy)-4-chloro-7α-(5-chloropentyl)-11β-fluoro-17β-methyloestra-1,3,5(10)-trien-17α-ol and 284 mg of (8,8,9,9,9-pentafluorononyl)methylamine as amine gave, after flash chromatography, 4-chloro-11β-fluoro-17β-methyl-7α-[5-[methyl (8,8,9,9,9-pentafluorononyl)amino]pentyl]oestra-1,3,5(10)-triene-3,17α-diol (176 mg).

H-NMR: 400 MHz, CDCl₃, δ=7.20 (d, 1H), 6.89 (m, 1H), 5.59 (d, 1H), 3.00 (d, 1H), 2.18 (s, 3H), 1.26 (s, 3H), 0.87 (d, 3H).

### Example 5

### 4-Bromo-11β-fluoro-17β-methyl-7α-[5-[methyl(8,8,9,9,9-pentafluorononyl)amino]pentyl]oestra-1,3,5(10)-triene-3,17α-diol

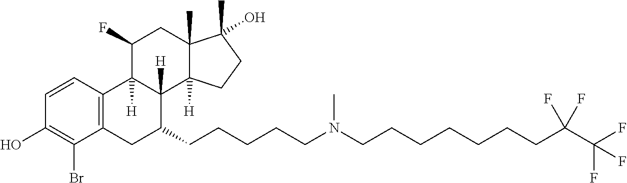

### 5.1 Preparation of the Intermediates

### 5.1.1 7α-(5-Chloropentyl)-11β-fluoro-17β-methyloestra-1,3,5(10)-triene-3,17α-diol

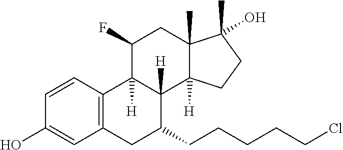

2.13 g of potassium fluoride and 0.55 ml of water were added successively to a solution of 3.2 g of 3-(tert-butyldimethylsilyloxy)-7α-(5-chloropentyl)-11β-fluoro-17β-methyloestra-1,3,5(10)-trien-17α-ol in 27 ml of ethanol, and the mixture was stirred at room temperature for 18 hours. The solvent was concentrated, and the residue was then taken up in ethyl acetate and washed 2× with water and sodium chloride solution. The organic phase was filtered and the solvent was removed on a rotary evaporator, and subsequent flash chromatography on silica gel (hexane/ethyl acetate) then gave 7α-(5-chloropentyl)-11β-fluoro-17β-methyloestra-1,3,5(10)-triene-3,17α-diol (2.74 g).

H-NMR: 400 MHz, CDCl₃, δ=7.20 (d, 1H), 6.67 (m, 1H), 6.57 (s (br.), 1H), 5.60 (d, 1H), 4.71 (s, 1H), 3.51 (t, 2H), 2.92 (dd, 1H), 2.71 (d, 1H), 2.54 (dd, 1H), 0.88 (d, 3H).

### 5.1.2 4-Bromo-7α-(5-chloropentyl)-11β-fluoro-17β-methyloestra-1,3,5(10)-triene-3,17α-diol

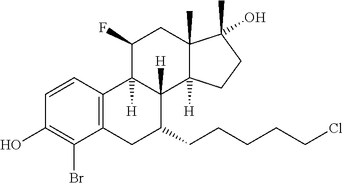

At 0° C., a solution of 1.17 g of N-bromosuccinimide in 167 ml of chloroform was added to a solution of 2.5 g of 7α-(5-chloropentyl)-11β-fluoro-17β-methyloestra-1,3,5(10)-triene-3,17α-diol in 278 ml of chloroform, and the mixture was then stirred at this temperature for 30 minutes. The mixture was then allowed to warm slowly to room temperature and stirred for a further 30 minutes. The reaction mixture was concentrated, and the residue was then taken up in ethyl acetate, washed 2× with water, dried over magnesium sulphate, filtered off and again concentrated to dryness. Flash chromatography on silica gel (hexane/ethyl acetate) gave 4-bromo-7α-(5-chloropentyl)-11β-fluoro-17β-methyloestra-1,3,5(10)-triene-3,17α-diol (1.86 g).

H-NMR: 400 MHz, CDCl₃, δ=7.25 (d, 1H), 6.91 (m, 1H), 5.60 (d, 1H), 5.56 (s, 1H), 3.52 (t, 2H), 2.95 (d, 1H), 1.26 (s, 3H), 0.87 (d, 3H).

### 5.2 Preparation of the End Product

At room temperature, 77.1 mg of sodium carbonate, 218.1 mg of sodium iodide and 270 mg of (8,8,9,9,9-pentafluorononyl)methylamine were added to a solution of 355 mg of 4-bromo-7α-(5-chloropentyl)-11β-fluoro-17β-methyloestra-1,3,5(10)-triene-3,17α-diol in 2.18 ml of dimethylformamide. The mixture was then stirred at 80° C. for 5 hours. After removal of the solvent, the residue was taken up in ethyl acetate and the solution was washed with water and semisaturated sodium bicarbonate solution, dried with magnesium sulphate and filtered off, and the solvent was removed on a rotary evaporator. Flash chromatography on silica gel (amine phase; hexane/ethyl acetate) gave 4-bromo-11β-fluoro-17β-methyl-7α-[5-[methyl(8,8,9,9,9-pentafluorononyl)amino]pentyl]oestra-1,3,5(10)-triene-3,17α-diol, which was recrystallized from diethyl ether (228 mg).

H-NMR: 400 MHz, CDCl₃, δ=7.24 (d, 1H), 6.90 (d, 1H), 5.59 (d, 1H), 2.96 (d, 1H), 2.18 (s, 3H), 1.26 (s, 3H), 0.87 (d, 3H).

### Example 6

### 4-Bromo-11β-fluoro-17β-methyl-7α-[5-[(2R)-2-(7,7,8,8,8-pentafluorooctyl)-1-pyrrolidinyl]pentyl]oestra-1,3,5(10)-triene-3,17α-diol

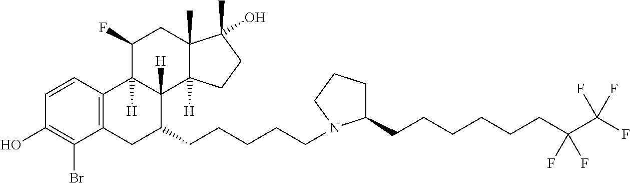

Analogously to step 5.2 of Example 5, 223 mg of 4-bromo-7α-(5-chloropentyl)-11β-fluoro-17β-methyloestra-1,3,5(10)-triene-3,17α-diol and 187 mg of (R)-2-(7,7,8,8,8-pentafluoro-octyl)pyrrolidine as amine (compound 2.1.1) gave, after flash chromatography (amine phase; hexane/ethyl acetate), 4-bromo-11β-fluoro-17β-methyl-7α-[5-[(2R)-2-(7,7,8,8,8-pentafluorooctyl)-1-pyrrolidinyl]pentyl]oestra-1,3,5(10)-triene-3,17α-diol (250 mg).

H-NMR: 400 MHz, CDCl₃, δ=7.24 (d, 1H), 6.90 (d, 1H), 5.60 (d, 1H), 3.13 (m, 1H), 2.96 (d, 1H), 1.26 (s, 3H), 0.86 (d, 3H).

### Example 7

### 4-Chloro-11β-fluoro-17β-methyl-7α-[5-[(2R)-2-(7,7,8,8,8-pentafluorooctyl)-1-pyrrolidinyl]pentyl]oestra-1,3,5(10)-triene-3,17α-diol

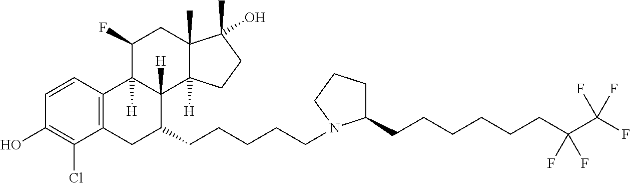

### 7.1 Preparation of the Intermediates

### 7.1.1 4-Chloro-7α-(5-chloropentyl)-11β-fluoro-17β-methyloestra-1,3,5(10)-triene-3,17α-diol

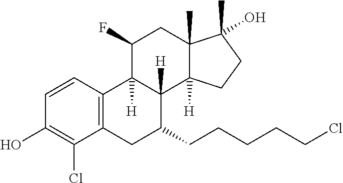

At room temperature, 164 mg of potassium fluoride and 42 μl of water were added successively to a solution of 262 mg of 3-(tert-butyldimethylsilyloxy)-4-chloro-7α-(5-chloropentyl)-11β-fluoro-17β-methyloestra-1,3,5(10)-trien-17α-ol in 8 ml of ethanol, and the mixture was stirred at room temperature for 24 hours. The solvent was concentrated, and the residue was then taken up in ethyl acetate and washed with water. After filtration of the organic phase and removal of the solvent on a rotary evaporator, subsequent flash chromatography on silica gel (hexane/ethyl acetate) gave 4-chloro-7α-(5-chloropentyl)-11β-fluoro-17β-methyloestra-1,3,5(10)-triene-3,17α-diol (94 mg).

H-NMR: 400 MHz, CDCl₃, δ=7.21 (d, 1H), 6.90 (d, 1H), 5.60 (d, 1H), 5.55 (s (br.), 1H), 3.52 (t, 2H), 2.98 (d, 1H), 1.26 (s, 3H), 0.87 (d, 3H).

### 7.2 Preparation of the End Product

Analogously to step 5.2 of Example 5, 206 mg of 4-chloro-7α-(5-chloropentyl)-11β-fluoro-17β-methyloestra-1,3,5(10)-triene-3,17α-diol and 209 mg of (R)-2-(7,7,8,8,8-pentafluorooctyl)pyrrolidine as amine (compound 2.1.1) gave, after repeated flash chromatography (silica gel followed by amine phase; hexane/ethyl acetate), 4-chloro-11β-fluoro-17β-methyl-7α-[5-[(2R)-2-(7,7,8,8,8-pentafluorooctyl)-1-pyrrolidinyl]pentyl]oestra-1,3,5(10)-triene-3,17α-diol (165 mg).

H-NMR: 400 MHz, CDCl₃, δ=7.20 (d, 1H), 6.90 (d, 1H), 5.60 (d, 1H), 3.12 (m, 1H), 2.99 (d, 1H), 1.26 (s, 3H), 0.876 (s (br.), 3H).

### Example 8

### 4-Bromo-11β-fluoro-17β-methyl-7α-[5-[(2R)-2-(6,6,7,7,7-pentafluoroheptyl)-1-pyrrolidinyl]pentyl]oestra-1,3,5(10)-triene-3,17α-diol

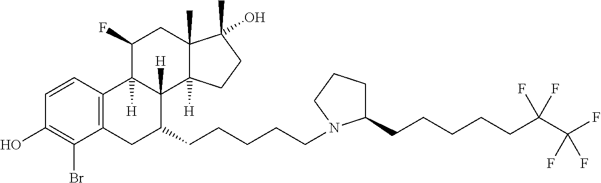

Analogously to step 5.2 of Example 5, 255 mg of 4-bromo-7α-(5-chloropentyl)-11β-fluoro-17β-methyloestra-1,3,5(10)-triene-3,17α-diol and 178 mg of (R)-2-(7,7,8,8,8-pentafluoro-heptyl)pyrrolidine as amine gave, after flash chromatography (silica gel: hexane/ethyl acetate), 4-bromo-11β-fluoro-17β-methyl-7α-[5-[(2R)-2-(6,6,7,7,7-pentafluoroheptyl)-1-pyrrolidinyl]pentyl]oestra-1,3,5(10)-triene-3,17α-diol (287 mg).

H-NMR: 400 MHz, CDCl₃, δ=7.24 (d, 1H), 6.90 (d, 1H), 5.60 (d, 1H), 3.12 (m, 1H), 2.96 (d, 1H), 1.26 (s, 3H), 0.86 (d, 3H).

### Example 9

### 4-Chloro-11β-fluoro-17β-methyl-7α-[5-[(2R)-2-(6,6,7,7,7-pentafluoroheptyl)-1-pyrrolidinyl]pentyl]oestra-1,3,5(10)-triene-3,17α-diol

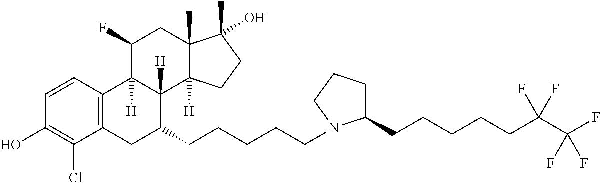

### 9.1 Preparation of Intermediates

### 9.1.1 4-Chloro-7α-(5-chloropentyl)-11β-fluoro-17β-methyloestra-1,3,5(10)-triene-3,17α-diol

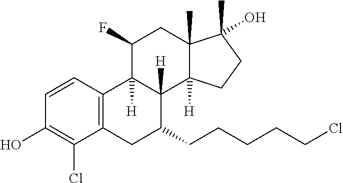

Alternatively to the procedure according to step 7.1.1 of Example 7, 4-chloro-7α-(5-chloropentyl)-11β-fluoro-17β-methyloestra-1,3,5(10)-triene-3,17α-diol could be prepared by adding, under an atmosphere of nitrogen, 239 mg of 2,3,4,5,6,6-hexachloro-2,4-cyclohexadien-1-one to a solution of 325 mg of (5-chloropentyl)-11β-fluoro-17β-methyloestra-1,3,5(10)-triene-3,17α-diol in 6 ml of dimethylformamide and stirring at room temperature for 24 hours. A further 12 mg of 2,3,4,5,6,6-hexachloro-2,4-cyclohexadien-1-one were then added. After a further 56 hours of stirring the reaction mixture was added to water and extracted with ethyl acetate. After drying and concentration of the organic phase, subsequent flash chromatography on silica gel (hexane/ethyl acetate) gave 4-chloro-7α-(5-chloropentyl)-11β-fluoro-17β-methyloestra-1,3,5(10)-triene-3,17α-diol (240 mg).

H-NMR: 400 MHz, CDCl₃, as stated under 7.1.1.

### 9.2 Preparation of the End Product

Analogously to step 5.2 of Example 5, 240 mg of 4-chloro-7α-(5-chloropentyl)-11β-fluoro-17β-methyloestra-1,3,5(10)-triene-3,17α-diol and 238 mg of (R)-2-(7,7,8,8,8-pentafluoro-heptyl)pyrrolidine as amine gave, after repeated flash chromatography (silica gel followed by amine phase; hexane/ethyl acetate), 4-chloro-11β-fluoro-17β-methyl-7α-[5-[(2R)-2-(6,6,7,7,7-pentafluoroheptyl)-1-pyrrolidinyl]pentyl]oestra-1,3,5(10)-triene-3,17α-diol (205 mg).

H-NMR: 400 MHz, CDCl₃, δ=7.20 (d, 1H), 6.89 (d, 1H), 5.60 (d, 1H), 3.14 (m, 1H), 2.99 (d, 1H), 1.26 (s, 3H), 0.87 (d, 3H).

### Example 10

### 2,4-Dichloro-11β-fluoro-17β-methyl-7α-[5-[(2R)-2-(7,7,8,8,8-pentafluorooctyl)-1-pyrrolidinyl]pentyl]oestra-1,3,5(10)-triene-3,17α-diol

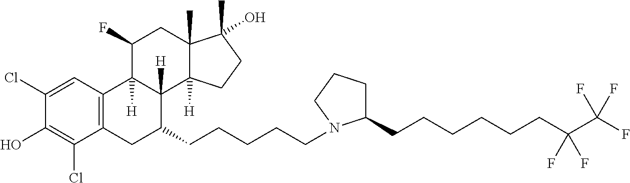

### 10.1 Preparation of the Intermediates

### 10.1.1 7α-(5-Chloropentyl)-2,4-dichloro-11β-fluoro-17β-methyloestra-1,3,5(10)-triene-3,17α-diol

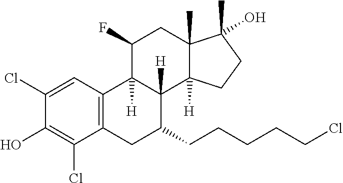

In addition to the desired pure target structure, the chlorination described in step 4.4.1 of Example 4 gave, after flash chromatography, also small amounts of the mixture of 3-(tert-butyldimethylsilyloxy)-4-chloro-7α-(5-chloropentyl)-11β-fluoro-17β-methyloestra-1,3,5(10)-trien-17α-ol and 3-(tert-butyldimethylsilyloxy)-7α-(5-chloropentyl)-2,4-dichloro-11β-fluoro-17β-methyloestra-1,3,5(10)-trien-17α-ol, which was desilylated according to the method described in Example 7.1.1. Flash chromatography on silica gel (hexane/ethyl acetate) gave 7α-(5-chloropentyl)-2,4-dichloro-11β-fluoro-17β-methyloestra-1,3,5(10)-triene-3,17α-diol (76 mg).

H-NMR: 400 MHz, CDCl₃, δ=7.28 (s, 1H), 5.82 (s (br.) 1H), 5.53 (d, 1H), 3.51 (t, 2H), 2.97 (d, 1H), 1.26 (s, 3H), 0.86 (s, 3H).

### 10.2 Preparation of the End Product

Analogously to step 5.2 of Example 5, 76 mg of 7α-(5-chloropentyl)-2,4-dichloro-11β-fluoro-17β-methyloestra-1,3,5(10)-triene-3,17α-diol and 65 mg of (R)-2-(7,7,8,8,8-pentafluoro-octyl)pyrrolidine as amine (compound 2.1.1) gave, after flash chromatography (silica gel; hexane/ethyl acetate) followed by prep. HPLC and the removal of traces of formic acid using sodium bicarbonate solution, 2,4-dichloro-11β-fluoro-17β-methyl-7α-[5-[(2R)-2-(7,7,8,8,8-pentafluorooctyl)-1-pyrrolidinyl]pentyl]oestra-1,3,5(10)-triene-3,17α-diol (16 mg).

H-NMR: 400 MHz, CDCl₃, δ=7.27 (s, 1H), 5.54 (d, 1H), 3.13 (m, 1H), 2.98 (d, 1H), 1.26 (s, 3H), 0.86 (d, 3H).

### B. PREPARATION OF COMPARATIVE 17α-ALKYL COMPOUNDS

### C1

### 11β-Fluoro-17α-methyl-7α-[5-[methyl(8,8,9,9,9-pentafluorononyl)amino]pentyl]oestra-1,3,5(10)-triene-3,17β-diol CAS [536975-64-1]

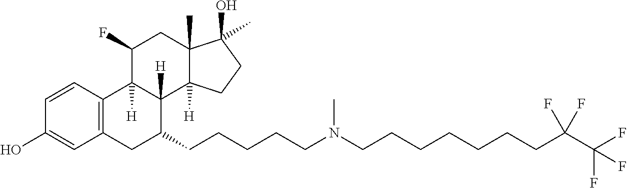

12.75 g of methyl-(8,8,9,9,9-pentafluorononyl)amine and 5.47 g of sodium carbonate were added to a solution of 19.5 g of 7α-(5-bromopentyl)-11β-fluoro-17α-methyloestra-1,3,5(10)-triene-3,17β-diol (WO 2003/045972, page 27, 2.2a) in 200 ml of absolute dimethylformaldehyde. The mixture was then stirred at 80° C. for 5.5 hours. After removal of the solvent, the residue was taken up in ethyl acetate, the mixture was washed with water and semisaturated sodium bicarbonate solution, dried with magnesium sulphate and filtered off and the solvent was removed on a rotary evaporator. Flash chromatography on silica gel (amine phase; hexane/ethyl acetate) gave 11β-fluoro-17α-methyl-7α-[5-[methyl(8,8,9,9,9-pentafluorononyl)amino]pentyl]oestra-1,3,5(10)-triene-3,17β-diol, which was recrystallized from diethyl ether (14.5 g);

H-NMR: 300 MHz, CDCl₃, δ=7.16 (d, 1H), 6.64 (d, 1H), 6.54 (d, 1H), 5.56 (d, 1H), 2.90 (d, 1H), 2.69 (d, 1H), 2.40-2.34 (m, 4H), 2.25 (s, 3H), 1.06 (d, 3H).

### C2

### 11β-Fluoro-17α-methyl-7α-[5-[(2R)-2-(7,7,8,8,8-pentafluorooctyl)-1-pyrrolidinyl]pentyl]-oestra-1,3,5(10)-triene-3,17β-diol

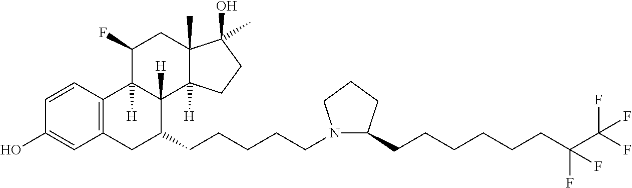

3.0 g of (2R)-2-(7,7,8,8,8-pentafluorooctyl)pyrrolidine, 2.01 g of sodium carbonate and 2.53 g of sodium iodide were added to a solution of 3.45 g of 7α-(5-chloropentyl)-11β-fluoro-17α-methyloestra-1,3,5(10)-triene-3,17β-diol in 45 ml of absolute dimethylformamide. The mixture was then stirred at 100° C. for 5 hours. After removal of the solvent, the residue was taken up in ethyl acetate, the mixture was washed with water and semisaturate sodium bicarbonate solution, dried with sodium sulphate and filtered off and the solvent was removed on a rotary evaporator. Flash chromatography on silica gel (amine phase; hexane/ethyl acetate) gave 11β-fluoro-17α-methyl-7α-[5-[methyl(8,8,9,9,9-pentafluorononyl)amino]pentyl]oestra-1,3,5(10)-triene-3,176-diol, which was recrystallized from diethyl ether (4.5 g).

H-NMR: 600 MHz, CDCl₃, δ=7.17 (d, 1H), 6.66 (d, 1H), 6.56 (d, 1H), 5.56 (d, 1H), 2.89 (d, 1H), 2.71 (d, 1H), 1.06 (d, 3H).

### C3

### 11β-Fluoro-17α-methyl-7α-[5-[(2R)-2-(6,6,7,7,7-pentafluoroheptyl)-1-pyrrolidinyl]pentyl]oestra-1,3,5(10)-triene-3,17β-diol

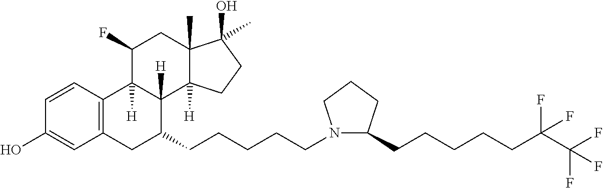

11.83 g of (2R)-2-(6,6,7,7,7-pentafluoroheptyl)pyrrolidine, 2.6 g of sodium carbonate and 7.33 g of sodium iodide were added to a solution of 10 g of 7α-(5-chloropentyl)-11β-fluoro-17α-methyloestra-1,3,5(10)-triene-3,17β-diol in 50 ml of absolute dimethylformamide. The mixture was then stirred at 80° C. for 18 hours. After removal of the solvent, the residue was taken up in ethyl acetate, the mixture was washed with water and semisaturated sodium bicarbonate solution, dried with sodium sulphate and filtered off and the solvent was removed on a rotary evaporator. Flash chromatography on silica gel (amine phase; hexane/ethyl acetate) gave 11β-fluoro-17α-methyl-7α-[5-[methyl(7,7,8,8,8-pentafluorononyl)amino]pentyl]oestra-1,3,5(10)-triene-3,17β-diol, which was recrystallized from diethyl ether (9.84 g).

H-NMR: 600 MHz, CDCl₃, δ=7.15 (d, 1H), 6.61 (d, 1H), 6.51 (d, 1H), 5.56 (d, 1H), 2.88 (d, 1H), 2.72 (d, 1H), 1.05 (d, 3H).

### C. CHARACTERIZATION

### C.1 Description of the Characterizing Experiments

### C.1.1 Metabolic Stability:

The metabolic stability of test substances was determined by incubations in a suspension with human liver microsomes adjusted to a protein content of 0.5 mg/ml, at a concentration of 0.3 μM. The incubation volume was 3.03 ml, where 2.4 ml of a microsome suspension in phosphate buffer at pH 7.4 (sodium phosphate buffer 100 mM (NaH₂PO₄×H₂O+Na₂HPO₄×2H₂O) were initially charged, which were activated by addition of 0.6 ml of a cofactor mix (consisting of 1.2 mg of NADP, 3 IU of glucose 6-phosphate dehydrogenase, 14.6 mg of glucose 6-phosphate and 4.9 mg of MgCl₂ in phosphate buffer, pH 7.4). The assay was started by addition of 30 μL of a test substance stock solution, where the composition of the stock solution was such that the solvent concentrations during the incubation were <0.2% for DMSO and <1% for methanol. The incubations were carried out at an incubation temperature of 37° C. over a period of 60 minutes, during which time the microsomes were kept in a homogeneous suspension by continuous stirring (Tec Control Shaker RS 485 at 300 rpm). At 6 different points in time (2, 8, 16, 30, 45 & 60 min), aliquots of in each case 250 μl were removed and immediately added to the same volume of ice-cold methanol and covered. The samples were frozen at −20° C. overnight and then centrifuged at 3000 rpm for 15 min, after which 100 μl of the clear supernatant were removed to determine the concentration. Analysis was carried out using an Agilent 1200 HPLC System coupled with LCMS/MS detection.

The decrease in concentrations over time were used to determine the half-life of the test substance in the microsomal incubation mixture described above, which in turn was used to calculate the “intrinsic clearance”, the maximum liver microsome rate for the metabolic elimination of the test substance. This “intrinsic clearance” for its part can be used together with various physiological parameters to estimate the maximum metabolic clearance in man, where the principle mentioned above is only capable of representing phase I metabolic reactions (typically: oxidoreductive reactions of Zytochrom P450 enzymes or flavin monooxigenase, and also hydrolytic reactions of esterases and amidases). Physiological parameters are: human liver blood flow: 1.3 l/kg/h; specific liver weight (per kg of body weight): 21 g/kg; microsomal protein content: 40 mg/g of liver. Furthermore, assuming that (i) the resorption of the test substance in humans is 100%, and (ii) the estimated metabolic clearance can be calculated as first pass, a maximum oral bioavailability (Fmax) was estimated.

Formulae & brief illustrations: CL intrinsic-apparent [in ml/(min*mg of protein)]: reflects the elimination constant kel (in min-1) of the test substance in the incubation mixture, divided by the microsomal protein content (40 mg/ml). CL intrinsic [in l(h*kg)]: maximum capability of the liver (microsomes) for metabolic elimination of the test substance, unless the liver blood flow is the limiting factor (kel*liver weight)/liver proportion (microsomal content) in the incubation. CL blood-well stirred [in l/(h*kg)]: estimated blood clearance (via Phase 1 metabolism): (QH*CL intrinsic)/(QH+CL intrinsic). Fmax [in %]: maximum bioavailability after oral administration of the test substance: (1-CL blood-well stirred/QH)*100.

### C.1.2 Bioavailability

The intragastral bioavailability of test substances was determined in female awake dogs having a body weight of at least 5 kg and at most 12 kg. To this end, the test substances were, both for the intravenous 15-min-infusion and for intragastral administration, administered in dissolved form, with compatible solubilizers such as PEG400 and/or ethanol being used in a compatible amount.

### Intravenous Administration:

At a low dose of 0.2-1 mg/kg, the test substances were infused over a period of 15 min. At the points in time 5 min, 10 min, 15 min (for example briefly before the end of the infusion), 20 min, 30 min, 45 min, 1 h, 2 h, 4 h, 6 h, 8 h, 24 h (based on the start of the infusion), blood samples of about 1-1.5 ml were taken. The blood samples were stored without shaking in lithium/heparin tubes (Monovettes® from Sarstedt) and centrifuged at 3000 rpm for 15 min. An aliquot of 100 μl was removed from the supernatant (plasma water) and precipitated by addition of 400 μl of cold ACN. The precipitated samples were frozen at −20° C. overnight and then centrifuged at 3000 rpm for 15 min, after which 150 μl of the clear supernatant were removed to determine the concentration. Analysis was carried out using an Agilent 1200 HPLC system with coupled LCMS/MS detection. Calculation of the PK parameters (via PK calculations software, for example WinNonLin®): CLplasma: total plasma clearance of the test substance (in l/kg/h); CLblood: total blood clearance of the test substance (in l/kg/h), where (CLblood=CLplasma*Cp/Cb); Vss: apparent distribution volume at steady state (in l/kg); T1/2: half-life within a specified interval (here: terminal T1/2, in h); AUCnorm: area under the plasma concentration time profile extrapolated from the point in time 0 to infinity divided by the dose (in kg*l/h); AUC(O-tn)norm: integrated area under the plasma concentration time profile from the point in time 0 to the last point in time where there was a measurable plasma concentration, divided by the dose (in kg*l/h); Cmax: maximum concentration of the test substance in plasma (in μg/l); Cmax,norm: maximum concentration of the test substance in plasma divided by the dose (in kg/l); Cb/Cp: ratio of the blood:plasma concentration distribution.

### Intragastral Administration:

At a low dose of 1-2 mg/kg, the test substances were administered intragastrally via stomach tubes as a bolus to unfed female dogs. At the points in time 15 min, 30 min, 45 min, 1 h, 2 h, 4 h, 6 h, 8 h, 24 h (based on the start of the infusion), about 1-1.5 ml of blood were removed. The blood samples were stored without shaking in lithium-heparin tubes (Monovettes® from Sarstedt) and centrifuged at 3000 rpm for 15 min. An aliquot of 100 μl was removed from the supernatant (plasma water) and precipitated by addition of 400 μl of cold ACN. The precipitated samples were frozen at −20° C. overnight and then centrifuged at 3000 rpm for 15 min, after which 150 μl of the clear supernatant were removed to determine the concentration. Analysis was carried out using an Agilent 1200 HPLC system with coupled

### LCMS/MS Detection

Calculation of the PK parameters (via PK calculation software, for example WinNonLin®): AUCnorm: area under the plasma concentration time profile extrapolated from the point in time zero to infinity divided by the dose (in kg*l/h); AUC(O-tn)norm: integrated area under the plasma concentration time profile from the point in time zero to the last point in time where there was a measurable plasma concentration, divided by the dose (in kg*l/h); Cmax: maximum concentration of the test substance in the plasma (in μg/l); Cmax,norm: maximum concentration of the test substance in the plasma divided by the dose (in kg/l); T1/2: half-life within a specified interval (here: terminal T1/2, in h); Fobs %: observed oral bioavailability, AUC(O-tn)norm after i.e. administration divided by AUC(O-tn)norm after i.e. administration. tmax: point in time where the maximum concentration of the test substance in the plasma is measured.

### C.2 Results

Table 2 shows the results of the PK characterization

(C1-3: direct analogues, i.e. they differ only in the stereoisomerism in position 17).

TABLE 2

| Compound [Ex.] | Fₘₐₓ (micros.) [%] | F_{obs} (Dog) [%] |
|---|---|---|
| 1 | 39 | 16 |
| C1 | 9 | 2.8 |
| 2 | 36 | 13 |
| C2 | 9 | 2 |
| 3 | 40 | 13 |
| C3 | 8 | 3.1 |
| 4 | 39 | |
| 5 | 55 | |
| 6 | 34 | |
| 7 | 47 | 14 |
| 8 | 48 | |
| 9 | 42 | |
| 10 | 34 | |

The compounds according to the invention having a 17β-group are superior to the direct analogues since they have increased metabolic stability in human microsomes and increased bioavailability in dogs after intragastral administration.

1. Compounds of the general formula (I) 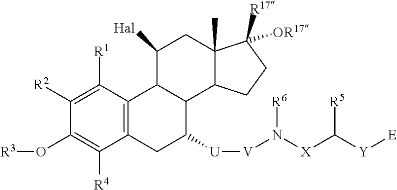
in which
Hal represents fluorine or chlorine which is attached in the 11β-position to the oestratriene skeleton; and
R¹, R² and R⁴ independently of one another, represent hydrogen, fluorine, chlorine or bromine, and
R³ represents hydrogen or a C₁-C₄-alkyl or C₁-C₄-alkanoyl radical, and
R^{17′} represents hydrogen or a C₁-C₄-alkyl or C₁-C₄-alkanoyl radical,
R^{17″} represents an optionally mono- or polyfluorinated C₁-C₄-alkyl, C₂-C₄-alkenyl or C₂-C₄-alkynyl radical,
where R^{17′}—O is attached in the 17α-position and R^{17′} is attached in the 17β-position to the oestratriene skeleton; and
U represents a straight-chain or branched C₁-c₁₃-alkylene, C₂-C₁₃-alkenylene or C₂-C₁₃-alkynylene radical, or
represents the group A-B, where
A is attached to the oestratriene skeleton and is a benzylidene radical attached via —CH₂— to the oestratriene skeleton, is a phenylene radical or is a C₁-C₃-alkylene-phenylene radical attached via the alkyl group to the oestratriene skeleton and B is a straight-chain or branched C₁-C₁₃-alkylene, C₂-C₁₃-alkenylene or C₂-C₁₃-alkynylene radical and
where A and B may also be attached to one another via an oxygen atom,
V represents a methylene or a —C(O)— group and
X represents a bond or a C₁-C₃-alkylene group and
R⁵ represents hydrogen or a C₁-C₄-alkyl, C₂-C₄-alkenyl or C₂-C₄-alkynyl radical
R⁶ represents hydrogen or the group —CH₂—R⁷ or C(O)—R⁷, in which R⁷ represents hydrogen or a straight-chain or branched nonfluorinated or at least partially fluorinated C₁-C₆-alkyl, C₂-C₆-alkenyl or C₂-C₆-alkynyl radical which may be mono- or polysubstituted by hydroxyl, or
R⁵ and R⁶ together with X and the nitrogen atom of the side chain form a 4- to 6-membered heterocyclyl ring which, in addition to the nitrogen atom of the side chain, may have a further heteroatom and/or may contain a carbonyl group,
Y represents a C₅-C₈-alkylene group,
E represents a C₁-C₄-perfluoroalkyl radical or represents a phenyl radical which is mono- to pentasubstituted by halogen or —CF₃,
and their enantiomers and diastereomers, their salts, solvates and salts of the solvates.

2. Compounds according to claim 1 in which
Hal represents fluorine and
R¹, R² und R⁴ independently of one another represent hydrogen, chlorine or bromine, and
R³ represents hydrogen, a methyl or acetyl radical, and
R⁵ represents hydrogen or a C₁-C₃-alkyl radical, and
R⁶ represents hydrogen or —CH₂—R⁷, in which R⁷ represents hydrogen or a methyl or ethyl radical, or
R⁵ und R⁶ together with the nitrogen atom of the side chain form a 5-membered heterocyclyl ring which, in addition to the nitrogen atom of the side chain, may have a further heteroatom and/or may contain a carbonyl group, and
R^{17′} represents hydrogen, a methyl radical or acetyl radical, and
R^{17″} represents a methyl, ethynyl or trifluoromethyl radical, and
U represents a butylene, pentylene, hexylene or heptylene radical, and
V represents a methylene group, and
X represents a bond or a methylene group, and
Y represents a bond or a C₅-C₇-alkylene group, and
E represents —C₂F₅, —C₃F₇, —C₄F₉ or represents a trifluoromethylphenyl radical,
and their enantiomers and diastereomers, their salts, solvates and salts of the solvates.

3. Compounds according to claim 1 in which
R¹ represents hydrogen, and
R² represents hydrogen or chlorine, and
R⁴ represents hydrogen, chlorine or bromine,
and their enantiomers and diastereomers, their salts, solvates and salts of the solvates.

4. Compounds according to claim 1 in which
R³ represents hydrogen,
and their enantiomers and diastereomers, their salts, solvates and salts of the solvates.

5. Compounds according to claim 1 in which
Hal represents fluorine,
and their enantiomers and diastereomers, their salts, solvates and salts of the solvates.

6. Compounds according to claim 1 in which represents hydrogen and R⁶ represents a methyl group or
R⁵ and R⁶ together with the nitrogen atom of the side chain form a pyrrolidine ring,
and their enantiomers and diastereomers, their salts, solvates and salts of the solvates.

7. Compounds according to claim 1 in which
R^{17′} represents hydrogen and R^{17″} represents a methyl radical,
and their enantiomers and diastereomers, their salts, solvates and salts of the solvates.

8. Compounds according to claim 1 in which
U represents an n-butylene radical:
and their enantiomers and diastereomers, their salts, solvates and salts of the solvates.

9. Compounds according to claim 1 in which
V represents a methylene group,
and their enantiomers and their diastereomers, their salts, solvates and salts of the solvates.

10. Compounds according to claim 1 in which
X represents a bond,
and their enantiomers and diastereomers, their salts, solvates and salts of the solvates.

11. Compounds according to claim 1 in which
Y represents an n-pentylene or n-hexylene group,
and their enantiomers and diastereomers, their salts, solvates and salts of the solvates.

12. Compounds according to claim 1 in which
E represents —C₂F₅,
and their enantiomers and diastereomers, their salts, solvates and salts of the solvates.

13. Compounds according to claim 1 in which
R¹ represents hydrogen, and
R² represents hydrogen or chlorine, and
R⁴ represents hydrogen, chlorine or bromine, and
R³ represents hydrogen, and
Hal represents fluorine, and
R⁵ represents hydrogen and R⁶ represents a methyl group or
R⁵ and R⁶ together with the nitrogen atom of the side chain form a pyrrolidine ring, and
R^{17′} represents hydrogen, and
R^{17″} represents a methyl radical, and
U represents an n-butylene radical, and
V represents a methylene group, and
X represents a bond, and
Y represents an n-pentylene or n-hexylene group, and
E represents —C₂F₅,
and their enantiomers and diastereomers, their salts, solvates and salts of the solvates.

14. Intermediates of the formula (IX) 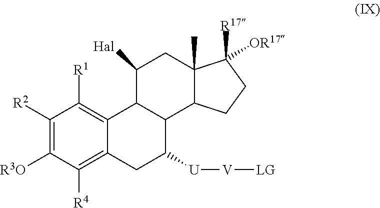
in which R¹, R², R³, R⁴, Hal, R^{17′}, R^{17″}, U and V have the meanings according to claim 1 and LG represents a sulphonate group or halogen.

15. (canceled)

16. (canceled)

17. Pharmaceutical formulation, comprising a compound according to claim 1 and a pharmaceutically acceptable excipient.

18. A method of treating an oestrogen-dependent disorder comprising administering to a human in need thereof a therapeutically acceptable amount of a compound according to claim 1.
